(19) United States
(12) Patent Application Publication
(10) Pub. No.: US 2008/0051660 A1
(43) Pub. Date: Feb. 28, 2008

(54) METHODS AND APPARATUSES FOR MEDICAL IMAGING

(75) Inventors: Ioannis A. Kakadaris, Bellaire, TX (US); Morteza Naghavi, Houston, TX (US); Sean M. O'Malley, Mountain View, CA (US)

Correspondence Address: ROBERT W STROZIER, P.L.L.C, PO BOX 429, BELLAIRE, TX, 77402-0429, US

(73) Assignee: THE UNIVERSITY OF HOUSTON SYSTEM, Houston, TX (US)

(21) Appl. No.: 11/833,759

(22) Filed: Aug. 3, 2007

Related U.S. Application Data
(63) Continuation-in-part of application No. 10/586,020.
(60) Provisional application No. 60/849,262, filed on Oct. 4, 2006.
(60) Provisional application No. 60/536,807, filed on Jan. 16, 2004.

(51) Int. Cl. A61B 8/06

(52) U.S. Cl. 600/454

(57) ABSTRACT

A set of intravascular ultrasound (IVUS) related systems, apparatuses and methods are disclosed. New catheter designs including contrast agent introduction subsystems and/or Doppler subsystems are disclosed. Methods for acquiring and analyzing Doppler data from intravascular ultrasound (IVUS) catheters are disclosed. RF-based detection of blood and/or contrast agents such as micro-bubbles are disclosed. Methods for frame-grating image data analysis permitting frame registration before, during and after a contrasting effect is imposed on a system being imaged are disclosed. Methods for difference imaging for contrast detection are disclosed. Methods for quantification and visualization of IVUS data are disclosed. And methods for IVUS imaging are disclosed.

### RELATED APPLICATIONS

This application is a continuation-in-part of U.S. patent application Ser. No. 10/586,020, filed Jul. 14, 2006, which is a 371 nationalized application of PCT Patent Application Ser. No. PCT/US05/01436, filed Jan. 14, 2005, which claims priority U.S. patent Provisional Patent Application Ser. No. 60/536,807, filed Jan. 16, 2004.

### GOVERNMENTAL INTEREST

Governmental entities may have certain rights in and to the contents of this application due to funded from NSF Grant IIS-0431144 and a NSF Graduate Research Fellowship (SMO).

### BACKGROUND OF THE INVENTION

1. Field of the Invention

The present invention relates to methods and systems for detecting and localizing vasa vasorum or other microvessels or micro-vascularizations associated with arteries, veins, tissues, organs and cancers in animals including humans.

More particularly, the present invention relates to methods and systems including the steps of acquiring contrast-enhanced data and analyzing the acquired data to prepare a view of the anatomy and/or morphology of portions of an artery, vein, tissue, organ and/or cancer within the scope of the acquired data evidencing micro-vascularizations. The present invention also relates to a set of inventions described in detail in specification section A-G. A. The present invention relates to new catheter designs including contrast agent introduction subsystems and/or Doppler subsystems. B. The present invention also relates to methods for acquiring and analyzing Doppler data from intravascular ultrasound (IVUS) catheters. C. The present invention also relates to method for RF-based detection and analysis of blood and/or contrast agents such as micro-bubbles from IVUS catheters operated in an RF mode. D. The present invention also relates to methods for frame-gating image data for enhanced IVUS imaging. E. The present invention also relates to methods for difference imaging in IVUS studies to enhance contrast detection. F. The present invention also relates to methods for quantification and visualization of vasa vasorum and parameters of risk in general based on vasa vasorum quantification. G. The present invention also relates to generalized methods for performing IVUS imaging studies.

2. Description of the Related Art

Contrast imaging is widely used in ultrasound imaging and other imaging formats to obtain enhanced information about a system such as a biological system. In biological system imaging, contrast imaging forms a basis for perfusion studies aimed at assessing blood flow through a region of vasculature or a particular organ. The contrast agents utilized in this context frequently include gaseous microbubbles contained in a stabilizing shell (diameter: 1-10 μm). These bubbles are designed to be efficient reflectors of incident ultrasound energy. Also blood and saline can be used as a contrasting agent in both static blood, saline or serum flow or in augmented or disrupted flow.

Intravascular ultrasound (IVUS) provides cross-sectional images of the interior of blood vessels at a high resolution. While a number of methods for computer-aided analysis of IVUS sequences have been proposed over the last decade, IVUS perfusion methods are more recent and less developed. This is because IVUS has traditionally been used as a tool for studying vessel morphology, which does not generally require the use of contrast. However, contrast-enhanced IVUS presents exciting opportunities for functional imaging.

Perfusion studies require that a particular anatomical region-of-interest be tracked over a period of time during which a contrast agent is introduced. In IVUS, while attempts are made to hold the imaging catheter (sensor) steady during recording, tracking is confounded by inter-frame motion variabilities, especially when imaging within the coronary arteries—heart and breathing rhythm variability of location of the sensor. Thus, there is a need in the art for a methodology that will permit frame tracking compensation for inter-frame motion variability.

### LAYOUT OF THE INVENTION

The present invention is divided into eight primary portions A-H. Each portion includes its own sections and own section numbering scheme. The reader is advised that each portion is self contained, except for figures. Figures are numbered independent of the portion of the application in which they appear.

### SUMMARY OF THE INVENTION

The present invention provides a method for medical imaging, where the method includes acquiring contrast-enhanced data and processing the acquired data to extract anatomical and/or morphological images of a body part being analyzed, where the method is well suited for producing anatomical, physiological (e.g., inflammation) and/or morphological data about a vessel including an extent of plaque development and/or inflammation and vasa vasorum associated with the vessel as well as anatomical and/or morphological data about structures within the detection scope of the method and where the body can be an animal including a human.

The present invention provides catheter for contrast enhanced IVUS (CEIVUS) and/or Doppler enhanced IVUS, where the catheters include a contrast agent delivery system and/or a Doppler sensor and a method for collecting Doppler data from a catheter.

The present invention provides a method for simultaneously performing IVUS imaging and Doppler blood flow imaging of regions of interest (ROIs) such as flow into and through sites of microvascularization such as vasa vasorum associated with a vessel being imaged. The Doppler imaging hardware is associated with the IVUS catheter so that only a single catheter or intra-arterial or intra-vascular device is required.

The present invention provides a radio-frequency (RF) detection and analysis methodology for blood, saline, microbubble and/or other contrast agents or contrast effects IVUS in both stationary-catheter and pullback catheter imaging.

The present invention provides frame-gating methods for stationary and pullback sequences.

The present invention provides a method for difference imaging analysis, where the method is adapted to detect and quantify regions of contrast perfusion into a vessel wall.

The present invention provides a method of visualizing micro-vascularized plaque (a plaque including vasa vasorum) and adventitia segments of a vessel in an animal or human body.

The present invention provides a method for imaging vulnerable plaque or other regions-of-interest (ROIs) using contrast enhanced IVUS imaging sometimes referred to herein as CEIVUS pronounced SEEVUS.

The present invention provides a method for visualizing vasa vasorum, where vulnerable plaque or other regions-of-interest (ROIs) are visualized using a radial segmentation technique.

### BRIEF DESCRIPTION OF DRAWINGS

The invention can be better understood with reference to the following detailed description together with the appended illustrative drawings in which like elements are numbered the same.

FIG. 1A-C depict an envelope image of computed IVUS signals, a log-compressed image of computed IVUS signals, and a geometrically transformed image of computed IVUS signals in the familiar disc-shaped IVUS image, respectively.

FIG. 2A-B depict SVM optimized over v and γ. Contour maps represent (a) blood true-positive rate and (b) support vector count. The marker at (γ=1, v=0.01) indicates a true-positive rate for blood f 97.1% and 101 support vectors. Similar plots (not shown) are made to show the false-positive rate in the plaque, in order to aid optimization.

FIG. 3A-B depict results for Ffi (first row of Table 1) overlaid on an (a) original frame and (b) its associated mask. Green: correctly classified as blood (as samples are located in the lumen); purple: correctly classified as non-blood (samples are located in the plaque); red: incorrectly classified as non-blood (samples are located in the lumen); yellow: classified as blood (while these are samples in the plaque, it is well known that small microvessels vasa vasorum grow in the plaque area). Whether the classification is correct will be determined by histology. Padding was added to the class boundaries in space and time (i.e., considering the previous and next frames in the sequence) to avoid skewing our results due to boundary effects, hence the classified points do not appear to fit the mask contours. In practice, every pixel in the image could be classified.

FIGS. 4A-B depict the first frame of a pullback sequence and a longitudinal slice through the stacked pullback volume, respectively. The “start” and “end” points of the line in (a) correspond to the top and bottom of the slice. Performing a S-C contrast study results in an image similar to (b), with the exceptions that vessel wall features do not gradually change over time and there is a brief period of luminal echo-opacity due to contrast injection.

FIGS. 5A-B depict dissimilarity matrices from the first 300 frames of a pullback and the 500 frames surrounding the time-of-interest in a stationary-catheter contrast study, respectively. In FIG. 5A, note the gradually-changing features overtime (i.e., from top-left to bottom-right); while in FIG. 5B note the contrast injection (bright bands) and how otherwise the matrix's features remain stable over time.

FIGS. 6A-C depict a dissimilarity matrix for the first 100 frames of a typical pullback sequence, along with dynamic-programming path (dotted line), a c function for the same matrix, and the matrix \hat{D} derived from the data of FIG. 1A, the same dynamic-programming path (dotted), the origin of the stepping process (Δ) along with associated steps (→), and the final frame pairs representing the gated sequence (Δ,∘).

FIGS. 7A-D depict phase histograms (number of frames selected per fraction of cardiac phase) for each of four cases, where the y-axes are normalized for comparability.

FIGS. 8A-C depict ungated pullback data, ECG-gated pullback data, and pullback data gated by the method of this invention. Differences in appearance between the latter two images are primarily due to their being captured at a different fraction of cardiac phase.

FIGS. 9A-B depict frame-similarity space clustered with k=3 and k=5, respectively, where the number of visible points in these plots was reduced to 184 to render the plots easier to interpret.

FIG. 10 depicts a trajectory plot of the frame-similarity space associated with FIG. 6B, along with high-level annotation, where adjacent frames are connected by a line.

FIG. 11 depicts improvement in mean inter-frame cross-correlation before and after stationary-catheter gating, for 12 human cases. The mean number of frames in the ungated sequences is 338±80.5; in the gated sequences, 103±16.5, where already-stable sequences show less signal improvement using the methods of this invention, while high-motion (low correlation) sequences show much higher degrees of improvement.

FIGS. 12A-D depict panels 12A and 12B are frames nearest cluster centroids for the first two clusters found with k=3 as shown in FIGS. 13C&D. Panels 12A and 12B represent images of two locations occupied by the imaging catheter and frequently imaged over the course of the contrast sequence. Panels 12C and 12D depict frames representing two outliers nearest a bottom of the frame set shown in FIG. 13B. Panel 12C and 12D These were captured at the peak of contrast agent density in the bloodstream, visible as a cloud around the catheter.

FIGS. 13A-F depict an analysis of a 184-frame sequence: panels 13A and 13B represent the original 184×184 matrix D and 2-D projection of frame-similarity space; panels 13C and 13D represent the same frame sequence clustered with k=3; and panels 13E and 13F represent the same frame sequence clustered with k=5, where dark points in the matrices indicate similar frames.

FIGS. 14A-C depict frames from a typical contrast sequence where panel 14A is a before image, panel 14B is a during image, and panel 14C is an after image, relative to contract agent injection.

FIGS. 15A-B depict extracting a swath, delineated by dotted lines, along a path (thick oval), where the origin in this case is at the center, while the arrows indicated the orientation of three columns extracted from the swath and the unwrapped parameterization of the 2-D swath image, where p=(w−1)/2, respectively.

FIG. 16 depict points along the discretized active contour are constrained to move only toward and away from the catheter during the elastic-matching phase (ID:ND).

FIG. 17A-B depicts a static-image contour and from top to bottom, the static-image swath, the moving-image swath after rigid but before elastic matching, the I¹ feature, and the feature with a path overlaid by an active contour, respectively, where perturbations from this being a horizontal line indicate inward/outward deviations from the original rigid contour. Note that in this representation, an upward deviation indicates the moving-image swath should move downward, and vice-versa (ID:ND).

FIG. 18 depicts the behavior of I³ as a function of the probability ratio defined by P. for a particular grey level g.

FIG. 19A-B depict an IVUS frame and its 5-region mask used for contour tracking, respectively.

FIGS. 20A-B depict an IVUS sequence before and after the introduction of contrast, respectively.

FIGS. 20C-D depict the plaque regions of these images before and after contrast; these regions have been registered using the contour tracking framework, respectively.

FIGS. 20E-F depict the raw and variance-modeled difference images obtained by subtracting FIG. 20C from FIG. 20D, respectively, where changes due to the introduction of contrast are exposed and are contrast-stretched for illustration.

FIG. 21 depicts a plot of a falloff in detected signal enhancement after introduction a contrast agent or contrast event in a vessel being imaged using an IVUS probe.

FIG. 22A-B depicts plaque and adventitia each divided into 4 quadrants, segmenting the images into 8 parts.

FIG. 23A-B depicts vasa vasorum (VV) density as assessed by histology and in vivo imaging allows comparison and validation.

FIGS. 24A-F the method for mapping plaque, adventitia and vasa vasorum (VV) density within quadrants of a segments of FIG. 23A, proximal, mid and distal.

FIGS. 25A-B depict 12-sector maps of plaque vasa vasorum (VV) density, where high vasa vasorum (VV) density in a proximal plaque and adventitia, respectively.

FIGS. 26A-B depict 12-sector maps of plaque vasa vasorum (VV) density, where high vasa vasorum (VV) density in a proximal plaque and adventitia, respectively, in an unfolded presentation.

### DETAILED DESCRIPTION OF THE INVENTION

The inventors have developed a number of systems, apparatuses and method for improving data derived from intravascular ultrasound. These systems, apparatuses and methods include: (A) new catheter designs including contrast agent introduction subsystems and/or Doppler subsystems; (B) methods for acquiring and analyzing Doppler data from intravascular ultrasound (IVUS) catheters; (C) method for RF-based detection of blood and/or contrast agents such as micro-bubbles; (D) methods for frame-grating image data analysis, (E) methods for difference imaging for contrast detection; (F) methods for quantification and visualization; and (G) methods for performing IVUS imaging.

### A. Catheter Design

The present invention also relates to a contrast enhanced IVUS (CEIVUS) catheter and/or Doppler enhanced IVUS catheter. The catheter includes a nozzle system having exit holes disposed around its periphery, where the holes are adapted to direct jets of a contrast agent near, immediately proximate or immediately adjacent a portion of a vessel wall of a vessel to be imaged. The portion of the vessel wall to which the contrast agent is directed can be immediately adjacent an IVUS probe or the nozzle system can be located a desired distance upstream or downstream of the probe. The nozzle system is connected via a conduit to an external or internal contrast agent reservoir. A flow of contrast agent from the reservoir to the nozzle system through the conduit is controlled by at least one electronic flow controller and injector or pump. The controllers and injector or pump can either introduce the contrast agent in a bolus introduction or pulsated introduction (a series of short pulses). The controller(s) is(are) in turn controlled by a digital or analog processing unit. The contrast agent can be blood, saline, microbubbles or any other contrast agent or contrast effect capable of inducing a detectable change in the imaged vessel portion or region of interest.

The catheters are designed to optimize contrast agent deliver so that high quality contrast images can be derived from contrast agent injection, especially to maximize the uptake of contrast agent into the vasa vasorum or other micro-vascularized structures in or associated with the vessel being imaged. The catheter may also include transducers irradiating at different frequencies for better contrast detection. The catheter can also be optimized for harmonic imaging—second and higher order effects and can include lock-in amplifiers and lock-in detectors for improved signal-to-noise. For further details on harmonic IVUS signal processing the reader is referred to WO2006/015877 A1, incorporated herein by reference.

The catheters are designed to include elements that permit Doppler data collection and method that allow Doppler data analysis. The Doppler elements and contrast delivery elements can be combined into a single catheter to permit contrast enhanced imaging and Doppler imaging to occur concurrently. The catheters can also include a separate IVUS probe or the catheters can include an IVUS probe, a nozzle system and a Doppler probe. The location of the system and probes are a matter of design preference and the type of data needed or desired.

### B. IVUS Doppler Studies of Atherosclerosis Plaque

Method for Doppler Imaging

The present invention also relates to a method for simultaneously performing IVUS imaging and Doppler blood flow imaging of regions of interest (ROIs) such as flow into and through sites of microvascularization such as vasa vasorum associated with a vessel being imaged. The Doppler imaging hardware is associated with the IVUS catheter so that only a single catheter or intra-arterial or intra-vascular device is required.

For rotating IVUS catheters, IVUS catheters including a single transducer subject to rotation about the vessel axis for whole vessel imaging, Doppler imaging is performed during periods at which the transducer or sensor is at rest. The at rest orientation would be selected so that the sensor is directed toward a ROI in the vessel such as a location of a micro-vascularized site (e.g., vasa vasorum etc.). Proper orientation may require manual rotation of the sensor or the catheter probe can include a controller to control an orientation of the sensor relative to a zero position. Once the sensor is at rest and properly oriented, Doppler imaging is performed. The Doppler IVUS imaging can be performed with or without a contrast agent. Blood, blood cells or saline can be used as the flow agent agents flowing through a structure.

For multi-sensor IVUS catheters, the method only includes Doppler imaging from one or all of the sensors depending on type of microvascularization structure being imaged.

Thus, the method includes the step of position a Doppler enchanced IVUS catheter is a vessel to be imaged. Once in place, IVUS images are collected. If the images are associated with a pull back study, then the IVUS catheter is pull back as images of the vessel are collected along the pull back path. Once a region of interest is detected, the catheter can be repositioned to that site and Doppler images acquired. The method can also include the step of injection a contrast agent. After contrast agent invention, IVUS images can be collected or Doppler images can be collected or a combination of IVUS and Doppler images offset by time can be collected. The method can include multiple contrast agent injections so that IVUS images and Doppler images can be collected separately and with sufficient dedicated injections.

Doppler Imaging

In the present invention also relates to a method including the step of after position of the probe, a few imaging pulses are transmitted into an ROI and echoes are received. The echoes are then matched or correlated echoes to the image to estimate the radial position of the stationary sensor on the image. Then, the sensor is switched to pulsed Doppler mode to look for flow signals at places in the image where a suspected plaque, microvascularization or vasa vasorum site is located. In addition, during Doppler measurements, an imaging pulse is transmitted periodically or intermittently to orient the Doppler beam and sample volume with respect to the image. If a slow rotational scan was used, then a color Doppler image can be constructed showing the location of vessels within the plaque. The magnitude and shape of the Doppler spectra and how it changes with or without the administration of contrast agents may provide information about plaque vulnerability. For further details on doppler imaging of blood flow in vessels, the reader is referred to U.S. Pat. Nos. 7,134,994; 7,097,620; 6,976,965; 6,962,567; 6,780,157; and 6,767,327, and as with all cited references as set forth in the last paragraph of the specification before the claims, these references are incorporated herein by reference.

### C. RF-Based Detection of Contrast Agent (Blood, Saline, Bubbles)

The present invention also relates to a radio-frequency (RF) detection, analysis and quantification methodology for IVUS in both stationary-catheter and pullback catheter imaging. The method includes the steps of obtaining RF-based IVUS data in a stationary catheter imaging study, where the stationary imaging can be performed with or without an external contrast agent. The method can also include the steps of obtaining RF-based IVUS data in a pullback catheter imaging study, where the imaging can be performed with or without an external contrast agent. In embodiments performed with contrast agent enhancement, the method includes the step of injecting a contrast agents or detecting natural flow of bodily fluid such as blood into the tissue being analyzed. Contrast agents or effects include blood, saline, serum, micro-bubbles, blood flow interruptions, blood flow augmentation, or the like.

The steps to perform RF-based detection of contrast perfusion into the vessel wall are described in the following text. For stationary-catheter contrast imaging, the catheter is positioned at the maximally-stenotic point of a suspect plaque and RF recording is performed before, during, and after injection of contrast agent (identical protocol to difference imaging). For stationary-catheter blood imaging, no injection is performed and recording only needs to be done for 7-13 cardiac cycles. For stationary-catheter pullback imaging, RF IVUS is recorded for a complete pullback sequence.

Training—Software to Discriminate Between Blood and Contrast Agent

The operator selects regions of interest in several frames of the sequence which encompass the target of interest (i.e., blood, saline, bubbles, etc.). Features are associated with each pixel in these ROIs. These features are composed of the coefficients associated with multidimensional frequency transforms (e.g., Fourier or wavelet): one for each window around each pixel in the region of interest. These windows will be 3-D, occupying multiple lines, samples, and frames (i.e., the third dimension is time). Given the coefficients and labels associated with each pixel, a learning algorithm is taught to distinguish between the feature of interest (blood/saline/serum/bubbles/etc.) and the background.

Deployment

For each frame in an unlabeled sequence, each pixel has a 3-D window of identical size to that used in training extracted around it, and the frequency-domain coefficients from each window are computed. These coefficients are given to the previously-trained learning algorithm, which provides the classification for each pixel.

Once each pixel in the sequence has been labeled, further processing may be performed to statistically quantify the presence of the feature of interest. For instance, bubble density per unit area or volume may be quantified, or the pullback sequence may be gated in order to produce a volumetric visualization of the analyzed frames.

One-Class Acoustic Characterization Applied to Blood Detection in IVUS

This portion of the specification describes a specific embodiment of an RF-based IVUS methodology. Intravascular ultrasound (IVUS) is an invasive imaging modality capable of providing cross-sectional images of the interior of a blood vessel in real time and at normal video frame rates (10-30 frames/s). However, obtaining a clear delineation between the blood surrounding the catheter and the vessel wall itself is a continuing problem in this field. As a result, various diagnostic procedures which rely on morphological statistics of the vessel are confounded and suffer from inter-observation variability. It would be beneficial therefore to have a method capable of detecting certain physical features, such as the blood, in an automated manner. We present an embodiment of a method for intravascular ultrasound capable of providing cross-sectional images. While blood detection algorithms are not new in this field, we deviate from traditional approaches to IVUS signal characterization in our use of 1-class learning. This eliminates certain problems surrounding the need to provide “foreground” and “background” (or, more generally, n-class) samples to a learner. Applied to the blood-detection problem on 40 MHz recordings made in vivo in swine, we obtain ≧98% sensitivity with ≧92% specificity at a radial resolution of ˜600 μm. The present invention provides contrast-free imaging of adventitial and intra-plaque blood: a critical capability for assessment of atherosclerotic plaque vulnerability. This is the first time a method has been presented capable of detecting extra-luminal blood.

### 1. INTRODUCTION

The majority of existing intravascular ultrasound (IVUS) systems rely on acoustic pulses generated at frequencies from 20 to 40 MHz. Going from lower to higher frequencies, we obtain higher-resolution images at the expense of decreased tissue penetration, greater noise, and greater backscatter from the blood. While the benefits obtained from improved image resolution often outweigh the other issues, the problem of blood backscatter is of particular concern as it makes it difficult for a human observer to distinguish the boundary between the blood and the vessel wall. This contributes to the known problems associated with the reproducibility of vessel morphology studies [1]. To help alleviate these problems, a number of computational methods have been developed over the last decade to detect blood in IVUS imagery [2, 3]. As these prior methods are primarily concentrated on the segmentation problem, they make little effort to detect blood beyond the luminal border. The method of this invention is capable of detecting extra-luminal blood. This opens the way to detecting the small vessels, vasa vasorum, that grow in the plaque, without contrast. The clinical importance of these vessels is their suspicion of being a main factor in atherosclerotic progression [4].

In this portion of the specification, the inventors develop methods to distinguish a single feature in medical imagery using 1-class learning techniques. In particular, we apply this to the problem of blood detection using IVUS techniques. The primary advantage the method disclose herein is that “background” samples need never be provided. The method derives the background from a wide variety of other imaged tissues. Providing suitable background samples may be labor-intensive and subjective. With 1-class learning, the method circumvents this problem entirely by ignoring background samples during training. Instead, training only requires samples of the foreground class which, in general, can be obtained relatively easily from expert annotations.

The problem of detecting intra-luminal blood is addressed here; however, this same technique can be readily extended to the problem of detecting blood elsewhere in the IVUS field of view. In this portion of the specification, the inventors have two goals: to describe how the recognizer framework may be applied to blood detection under ultrasound, and to examine specific features useful for accomplishing this. In Section 2, the inventors provide background on the problems surrounding our task. In Section 3, the inventors discuss our contribution. We conclude with our results set forth in Section 4 and a discussion set forth in Section 5.

### 2. BACKGROUND

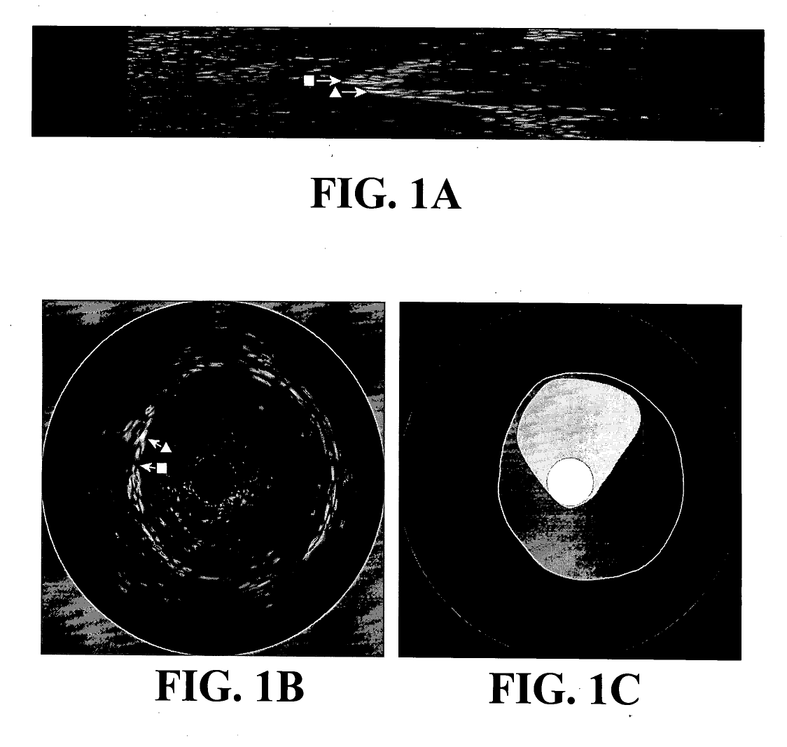

The intravascular ultrasound (IVUS) catheter consists of either a solid-state or a mechanically-rotated transducer which transmits a pulse and receives an acoustic signal at a discrete set of angles over each radial scan. Commonly, 240 to 360 such one-dimensional signals are obtained per (digital or mechanical) rotation. The envelopes of these signals are computed, log-compressed, and then geometrically transformed to obtain the familiar disc-shaped IVUS image see FIGS. 1A-C. However, most of our discussion will revolve around the original polar representation of the data. That is, stacking the 1-D signals we obtain a 2-D frame in polar coordinates. Stacking these frames over time, we obtain a 3-D volume I(r;θ;t) where r indicates radial distance from the transducer, 0 the angle with respect to an arbitrary origin, and t the time since the start of recording (i.e., frame number). The envelope and log-compressed envelope signals are represented by Iₑ and Iₗ respectively. Note that I contains real values while Iₑ and Iₗ are strictly non-negative. The I, signal represents the traditional method of visualizing ultrasound data, in which log compression is used to reduce the dynamic range of the signal in order for it to be viewable on standard hardware. This signal is the basis for texture-based characterization of IVUS imagery. The signal I has a large dynamic range and retains far more information, including the frequency-domain information lost during envelope calculation. This “raw” signal is the basis for more recent radiofrequency-domain IVUS studies.

Referring now to FIG. 1A, a log-compressed envelope of the IVUS signal in polar format is shown. The r-axis is horizontal (the origin being at the left, at the catheter) and the axis vertical (of arbitrary origin). Looking at FIG. 1B, the same signal after Cartesian transformation is shown. The arrows marked 4 and (provided for orientation only) are positioned similarly in the polar and Cartesian spaces. Looking at FIG. 1C, a diagram of the features of interest is shown, from the center outward: catheter, blood, plaque, and adventitia and surrounding tissues.

One-class Learning

The backbone of our method is the 1-class support vector machine (SVM); a widely-studied 1-class learner or “recognizer.” The problem of developing a recognizer for a certain class of objects can be stated as a problem of estimating the possibly high-dimensional (PDF) of the features characterizing those objects, then setting a probability threshold which separates in-class objects from all other out-of-class objects. This threshold is necessary since, as learning does not make use of out-of-class examples, the in-class decision region could simply cover the entire feature space, resulting in 100% true- and false-positive rates. Following the approach of Schölkopf et al [5], we denote this threshold as vε(0,1). We note that as the learner is never penalized for false positives (due to its ignorance of the negative class), it is essential that the PDF's of the positive and negative classes are well-separated in the feature space.

The other parameter of interest is the width function of the SVM radial basis function (i.e., k(x,x′)=exp(−γ∥x−′∥²) for a pair of feature vectors x and x′). Properties of a good SVM solution include an acceptable classification rate as well as a low number of resulting support vectors. A high number of support vectors relative to the number of training examples is not only indicative of overfitting, but is computationally expensive when it comes to later recognizing a sample of unknown class. A further discussion of the details of SVM operation is outside the scope of this application; the interested reader is encouraged to consult the introduction by Hsu et al [6].

### 3. MATERIALS & METHODS

3.1 Data Acquisition and Ground Truth

Ungated intravascular ultrasound sequences were recorded at 30 frames/s in vivo in the coronary arteries of five atherosclerotic swine. The IVUS catheter's center frequency was 40 MHz. Each raw digitized frame set I(r;θ;t) consists of 1794 samples along the r axis, 256 angles along the θ axis, and a variable number of frames along t (usually several thousand). The envelope Iₑ and log-envelope Iₗ signals were computed offline for each frame.

For training and testing purposes, a human expert manually delineates three boundaries in each image: one surrounding the IVUS catheter, one surrounding the lumen, and one surrounding the outer border of the plaque as shown in FIG. 1C. The blood within the lumen is used as the positive class in training and testing. As our goal is to separate blood from all other physical features, we use the relatively blood-free tissue of the plaque as the negative class in testing. For the purposes of this study we ignore the adventitia and surrounding tissues: they not only frequently contain free blood themselves, but are often very difficult for a human observer to reproducibly interpret.

3.2 Features

We analyze two classes of features: those intended to quantify speckle (i.e., signal randomness in space and time) and those based on frequency-domain spectral characterization. The former are traditionally used for blood detection and the latter for tissue characterization. These features are defined for a 3-D signal window of dimensions r_{θ}×θ_{θ}×t_{θ} to as follows:

$$\begin{matrix}{F}_{α} = \frac{1}{{r}_{0} {θ}_{0}} {\sum }_{i = 1}^{{r}_{0}} {\sum }_{j = 1}^{{θ}_{0}} stddev [ I ( i , j , \cdot ) ] & ( 1 ) \\ {F}_{β} = \frac{1}{{r}_{0} {θ}_{0} {t}_{0}} {\sum }_{i = 1}^{{r}_{0}} {\sum }_{j = 1}^{{θ}_{0}} {\sum }_{k = 1}^{{t}_{0}} \vert I ( i , j , k ) \vert & ( 2 ) \\ {F}_{δ} = \frac{1}{{r}_{0} {θ}_{0}} {\sum }_{i = 1}^{{r}_{0}} {\sum }_{j = 1}^{{θ}_{0}} corr [ I ( i , j , \cdot ) ] & ( 3 ) \\ {F}_{ξ} = \frac{1}{{t}_{0}} {\sum }_{k = 1}^{{t}_{0}} stddev [ I ( \cdot , \cdot , k ) ] & ( 4 ) \\ {F}_{ζ} = {\sum }_{i = 1}^{[ {r}_{0} / 2 ]} {\sum }_{j = 1}^{[ {θ}_{0} / 2 ]} {\sum }_{k = 1}^{[ {t}_{0} / 2 ]} ijk \hat{I} ( i , j , k ) & ( 5 ) \\ {F}_{η} = \frac{{F}_{ζ}}{{\sum }_{i = 1}^{[ {r}_{0} / 2 ]} {\sum }_{j = 1}^{[ {θ}_{0} / 2 ]} {\sum }_{k = 1}^{[ {t}_{0} / 2 ]} \hat{I} ( i , j , k )} & ( 6 ) \\ {F}_{t} = FFT \{ mean\_single [ I ] \} & ( 7 )\end{matrix}$$

where stddev(·) returns the sample standard deviation of the samples in its argument and corr(·) returns the correlation coefficient of its argument compared to a linear function (e.g., a constant signal), returning a value on [−1; +1]. The function Î indicates the magnitude of the Fourier spectrum of I. FFT(·) computes the magnitude of the Fourier spectrum of its vector input (the result will be half the length of the input due to symmetry) and mean_signal (·) takes the mean of the θt IVUS signals in the window, producing one averaged 1-D signal.

The features represent measures of temporal (F_{α} and F_{δ}) and spatial (F_{ε}) speckle, a measure of signal strength (F_{β}), measures of high-frequency signal strength (F_{ζ} and, normalized by total signal strength, F_{η}), and a vector feature consisting of the raw backscatter spectrum (Fₜ). In practice, this final feature is windowed to retain only those frequencies within the catheter bandwidth (˜20-60 MHz in our case). Each feature, with the exceptions of (F_{ζ}, F_{η}, Fₜ), are computed on Iₑ and Iₗ in addition to I. Hence, features (F_{α}, F_{β}, F_{δ}, F_{ε}) actually consist of vectors of three values. Feature (Fₜ) consists of a vector that varies according to the sampling rate and bandwidth of the IVUS system.

Samples are extracted by setting a fixed window size (r₀, θ₀, t_{θ}) and, from a set of consecutive IVUS frames (i.e., a volume) for which associated manually-created masks are available, placing the 3-D window around each sample in the volume. If this window does not overlap more than one class, the above features are computed for that window and associated with the class contained by it. To improve the scaling of the feature space, each feature of the samples used for training are normalized to zero mean and unit variance. The normalization values are retained for use in testing and deployment.

3.3 Training & Testing Scheme

In general, given a set of positive S₊ and negative S₋ samples (from the lumen and plaque respectively), which typically represent some subset of our seven features, a grid search over y and v is performed to optimize a one-class SVM. Optimization in this case aims to obtain an acceptable true positive rate on S₊, true negative rate on S₋, and low number of support vectors. In order to avoid bias, at every (γ; v) point on the grid, 5-fold cross-validation is used. That is, the recognizer is trained on one-fifth of S₊ and tested on the remaining four-fifths of S₊ and all of S₋ (the negative class is never used in training).

As feature selection is especially critical in a one-class training scenario, we gauge the performance of each feature individually. More elaborate feature selection schemes such as genetic algorithms [7] could be used, but as one of our goals here is to determine which feature(s) best characterize the blood, we will not investigate this issue.

### 4. RESULTS

For space reasons, we will analyze in detail the results from one typical case from our animal studies. (The results from additional cases are very similar due to their being recorded with the same IVUS hardware.) For each of our seven features, we will obtain the best possible results using the training method described previously. That is, we will choose the parameters v and y such that there is a true-positive rate (sensitivity) of, ≧98%, where possible, and a minimal false-positive rate. The number of support vectors at this point will be indicative of the generalization power of the feature. A final parameter to be mentioned is the window size for feature extraction. In previous experiments we determined an effective tradeoff between window size and spatial accuracy to be (r_{θ}; θ_{θ}; t_{θ})=(255; 13; 13); this equates to a radial resolution of ˜600 μm, angular resolution of ˜18°, and temporal resolution of ˜0.4 s. These values will vary by IVUS system but, in general, larger windows provide better classification at the expense of resolution. (Note that a temporal window of t_{θ}=13 may be excessively long for an IVUS system whose frame rate is below 30 frames/s.)

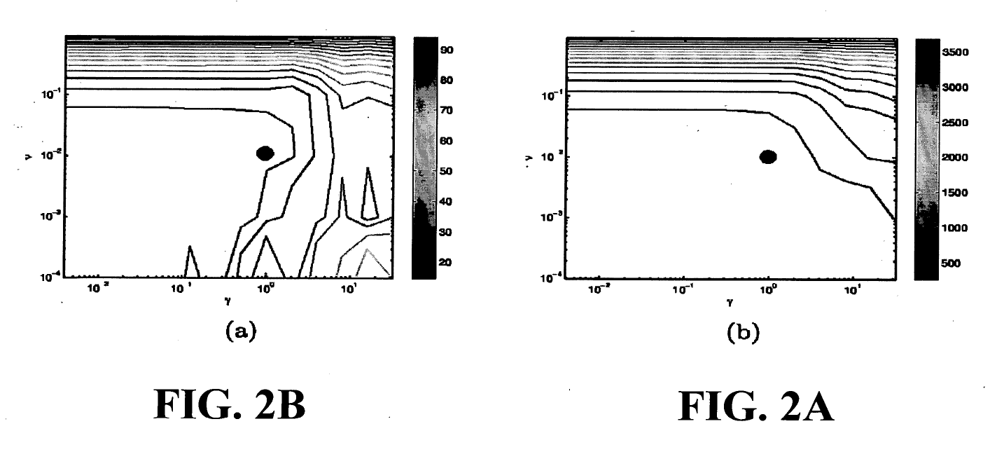

Referring now to FIG. 2, an SVM optimization over v and γ. Contour maps represent (a) blood true-positive rate and (b) support vector count. The marker at (γ=1, v=0.01) indicates a true-positive rate for blood of 97.1% and 101 support vectors. Similar plots (not shown) are made to show the false-positive rate in the plaque, in order to aid optimization.

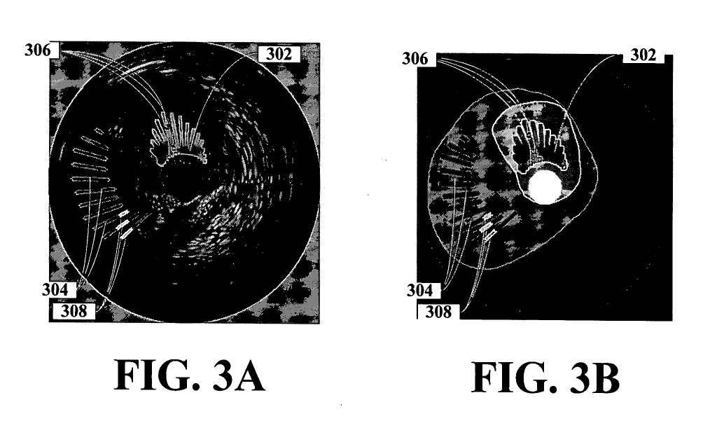

Table 1 summarizes the results for each feature for a typical sequence. To determine whether the performance of a particular feature was mainly due to that feature's application to a specific form of the data (i.e., either the raw signal, its envelope, or its log-compressed envelope), this table also lists the results of subdividing three of the highest-accuracy features into their components and performing experiments on these alone. Lastly, results on a typical frame are illustrated graphically in FIGS. 3A-B.

### 5. DISCUSSION AND CONCLUSION

Our highest performance was obtained using features which attempt to directly measure the amount of variability (“speckle”) present in the signal, either temporally (F_{α}), spatially (F_{ε}), or in the frequency domain (F_{ζ}, F_{η}). Direct learning from the Fourier spectrum tended to perform poorly (Fₜ). This is likely because one-class learning is ill-suited to determining the subtle differences in frequency spectra between the backscatter of various features imaged under ultrasound. The performance of these features as applied to a single signal type (e.g., F_{α}*) tended to be poorer than the result obtained otherwise (e.g., F_{α}). However, this trend does not extend to increased performance when a larger number of features are combined during training. For instance, we found that using all features except F_{ζ} together results in prohibitively poor specificity (<20%). This is an expected result for one-class SVMs, as their performance will degrade with the inclusion of features in whose spaces the objects of interest are poorly separated.

TABLE 1
Statistics Relating the Classification Accuracy

| Fea- | | | | | | | |
|---|---|---|---|---|---|---|---|
| ture | TP | FP | TN | TN | Sensitivity | Specificity | SV (%) |
| F_{α} | 8644 | 705 | 8334 | 93 | 98.9 | 92.2 | 106 (1.2) |
| F_{β} | 8727 | 3868 | 5171 | 10 | 99.9 | 57.2 | 17 (0.2) |
| F_{δ} | 8649 | 8796 | 243 | 88 | 99.0 | 2.69 | 102 (1.2) |
| F_{ε} | 8716 | 2 | 9037 | 21 | 99.8 | 100 | 33 (0.4) |
| F_{δ} | 8653 | 1264 | 7775 | 84 | 99.0 | 86.0 | 98 (1.1) |
| F_{η} | 8600 | 2334 | 6705 | 137 | 98.4 | 74.2 | 246 (2.8) |
| F_{ζ} | 5010 | 27 | 9012 | 3727 | 57.3 | 99.7 | 8083 (92.5) |
| F_{α}* | 8404 | 3446 | 5593 | 333 | 96.2 | 61.9 | 271 (3.1) |
| F_{α}^{†} | 8064 | 281 | 6228 | 673 | 92.3 | 68.9 | 391 (4.5) |
| F_{α}^{‡} | 8094 | 2552 | 6487 | 643 | 92.6 | 71.8 | 373 (4.3) |
| F_{ε}* | 7838 | 3488 | 5551 | 899 | 89.7 | 61.4 | 271 (3.1) |
| F_{ε}^{†} | 7623 | 2963 | 6077 | 1114 | 87.2 | 67.2 | 187 (2.1) |
| F_{ε}^{‡} | 7542 | 1860 | 7179 | 1195 | 86.3 | 79.4 | 191 (2.2) |
| F_{δ}* | 8576 | 3241 | 5798 | 161 | 98.2 | 64.1 | 163 (1.9) |
| F_{δ}^{†} | 8591 | 3264 | 7557 | 146 | 98.3 | 63.9 | 160 (1.8) |
| F_{δ}^{‡} | 8417 | 3277 | 5762 | 320 | 96.3 | 63.7 | 147 (1.7) |

Table 1. Statistics relating the classification accuracy obtained by each feature with respect to true/false (T/F) positives/negatives (P/N). Positive/negative examples used: 8737/9039. Sensitivity is defined as TP/(TP+FN); specificity as TN/(TN+FP). Support vectors (SV) are listed as an absolute value and as a percentage of the number of (positive) examples used for training. Also shown are statistics relating the classification accuracy obtained by features F_{α}, F_{ε}, and F_{ζ}; when they are applied to only one type of signal: the original*, envelope^{†}, and log-envelope^{‡}.

In the experiments described here, training and testing were performed on each sequence independently (though, with cross-validation, samples used in training were never used in testing). A topic of future investigation is whether a recognizer trained on one sequence will have similar accuracy when applied to another (for instance, a sequence recorded in a different subject). With histological aid, we will also determine the sensitivity of our approach when applied to the problem of detecting extra-luminal blood.

Referring now to FIGS. 3A-B, the results for F_{α} (first row of Table 1) overlaid on an original frame as shown in FIG. 3A and its associated mask as shown in FIG. 3B. Green 302: correctly classified as blood (as samples are located in the lumen); purple 304: correctly classified as non-blood (samples are located in the plaque); red 306: incorrectly classified as non-blood (samples are located in the lumen); yellow 308: classified as blood (while these are samples in the plaque, it is well known that small microvessels vasa vasorum grow in the plaque area). Whether the classification is correct will be determined by histology. Padding was added to the class boundaries in space and time (i.e., considering the previous and next frames in the sequence) to avoid skewing our results due to boundary effects, hence the classified points do not appear to fit the mask contours. In practice, every pixel in the image could be classified.

### 6. REFERENCES

The following references were cited in this portion of the specification:
1. Rodriguez-Granillo, G. A., McFadden, E. P., Aoki, J., van Mieghem, C. A. G., Regar, E., Bruining, N., Sermuys, P. W.: In vivo variability in quantitative coronary ultra-sound and tissue characterization measurements with mechanical and phased-array catheters. hit J Cardiovasc Imaging 22 (2006) 47-53
2. Pasterkamp, G., van der Heiden, M. S., Post, M. J., ter Haar Romeny, B. M., Mali, W. P. T. M., Borst, C.: Discrimination of the intravascular lumen and dissections in a single 30-MHz US image: Use of “confounding” blood backscatter to advantage. Radiology 187 (1993) 871-872
3. Hibi, K., Takagi, A., Zhang, X., Teo, T. J., Bonneau, H. N., Yock, P. G., Fitzgerald, P. J.: Feasibility of a novel blood noise reduction algorithm to enhance reproducibility of ultra-high-frequency intravascular ultrasound images. Circulation 102 (2000) 1657-1663
4. Gõssl, M., Versari, D., Mannheim, D., Ritman, E. L., Lerman, L. O., Lerman, A.: Increased spatial vasa vasorum density in the proximal LAD in hypercholesterolemia Implications for vulnerable plaque-development. Atherosclerosis (2006)
5. Schölkopf, B., Platt, J. C., Shawe-Taylor, J., Smola, A. J., Williamson, R. C.: Estimating the support of a high-dimensional distribution. Neural Comput 13 (2001) 1443-1471
6. Hsu, C. W., Chang, C. C., Lin, C. J.: A practical guide to support vector classification. Technical report, Dept. of Computer Science and Information Engineering, National Taiwan University (2004)
7. Yang, J., Honavar, V.: Feature subset selection using a genetic algorithm. IEEE Intell Syst App 13 (1998) 44-49

### D. Image-Based Gating of Pullback and Stationary-Catheter Sequences in Intravascular Ultrasound

Section I—Introduction

Intravascular ultrasound is an invasive, catheter-based imaging modality which provides cross-sectional images of the interior of a blood vessel in real time and at video frame rates. For studies of vessel morphology, the transducer-bearing catheter is gradually withdrawn through the vessel during recording in order to allow digital reconstruction of a 3-D volumetric image of the vessel. The inventors refer to these studies as “pullback” sequences. For functional imaging, the catheter is held stationary while recording, during which time an ultrasound contrast agent and/or other drugs may be introduced into the bloodstream. The inventors refer to the stationary phase of the studies as “stationary-catheter” (S-C) sequences. In both cases, motion artifacts relating to the beating heart and idiosyncrasies of the imaging protocol may render these sequences difficult to analyze without subsequent gating. A simple and generally effective way to account for these motions is to gate the sequences according to an electrocardiogram (ECG) signal. In essence, the electrical behavior of the heart is used as an indicator of its physical pose.

The inventors have found that a frame-gating technique can be constructed to alleviate a wide variety of periodic and non-periodic motion artifacts in a sequence of images acquired for a system, especially system that undergo contrast enhancement during the collection period, which generally extends from a first time t₁ before contract enhancement and a second time t₂ after contrast enhancement. Unlike previous efforts which either utilize ECG signals directly or attempt to mimic their performance through image analysis, the inventors have instead performed an appearance-based grouping of frames. In this way, unusual events (e.g., catheter slippage), common periodic effects (e.g., longitudinal catheter motion), and more subtle changes during recording (e.g., varying heart and breathing rates) are more implicitly and simply accounted for.

While one goal of the method is simply to extract a single stabilized subset of a longer frame sequence, by formulating the problem in terms of multidimensional scaling (MDS), a number of other useful operations may be performed. The MDS transform places points defined only by inter-point proximities into a metric space such that the proximities are preserved with minimal loss [2]. This preservation allows a creation of a frame-similarity space which is employed as a concise visual and numerical summary of an entire frame sequence. Clustering this space allows sets of frames with various similarity properties to be extracted efficiently. In addition, the method is self-calibrating in the sense that it need not be tuned to a grey-level, noise, or motion properties of the sequence at hand; e.g., the method can be applied in an identical manner to 20 MHz and 40 MHz IVUS data acquired in humans and swine with similar results compared to adjusting the criteria at each analysis. One aspect of the method is simply to obtain a sufficiently stable sequence. Therefore, the method does not require that frames be captured or acquired at a specific fraction of the cardiac cycle in order to register frames based on where in the cycle the image was acquired.

While ECG-based gating methods are simple to implement and have a long track record of use, they are potentially sub-optimal for image-stabilization purposes. Obviously, ECG-based gating also cannot be applied to sequences for which associated ECG signals were not recorded.

The inventors here introduce gating methods for both pullback and S-C sequences. The former emulates ECG, with the exception that it automatically selects the fraction of the cardiac cycle that provides an optimally stable frame set according to certain criteria. The latter clusters frames into related groups, ignoring the cardiac cycle, and as a side effect is able to produce a simple graphical depiction of the motion behavior of the entire sequence. In this way, the method of this invention is suitable for analyzing a wider variety of motion artifacts than is capable using ECG-based gating method; for instance, unintentional movement of the catheter during recording. One differences between the two methods is that one selects a single frame per cycle, the other potentially multiple frame per cycle due to the intended applications of the two methods: morphological versus functional imaging. Both methods are driven by the imaging data alone and do not require ECG data. In addition, as robust fully-automated algorithms for IVUS segmentation do not currently exist, these methods were developed so as to not require prior segmentation of the IVUS frames. Instead, the inventors rely on pair-wise frame comparisons, which is performed using common registration metrics.

This portion of the application is organized as follows. In Section II, the inventors discuss prior research in the field and in Section III, the inventors introduce our gating methods. In Section IV, the inventors validate our pullback gating method by comparing it to the performance obtained by standard gating with synchronously-recorded ECG. As there does not exist ground truth for S-C sequence gating, we compare the inter-frame stability properties of ungated versus gated S-C sequences. The inventors conclude in Section V.

Section II—Prior Methodologies

The use of ECG signals in medical imaging is ubiquitous as a means of stabilizing image sequences, which generally suffer from cardiac motion artifacts. As the features exhibited by this time-domain signal correspond closely to cardiac activity, ECG data is used as a non-invasive indicator of cardiac pose. The most apparent feature in this signal is the R-wave: due to its prominence. Thus, points or image frames in time during the cardiac cycle are typically referred to as a fraction of the interval between adjacent R-waves. Of importance is the fact that, in principal, the heart should be in roughly the same pose at each point in time corresponding to the same R-R fraction.

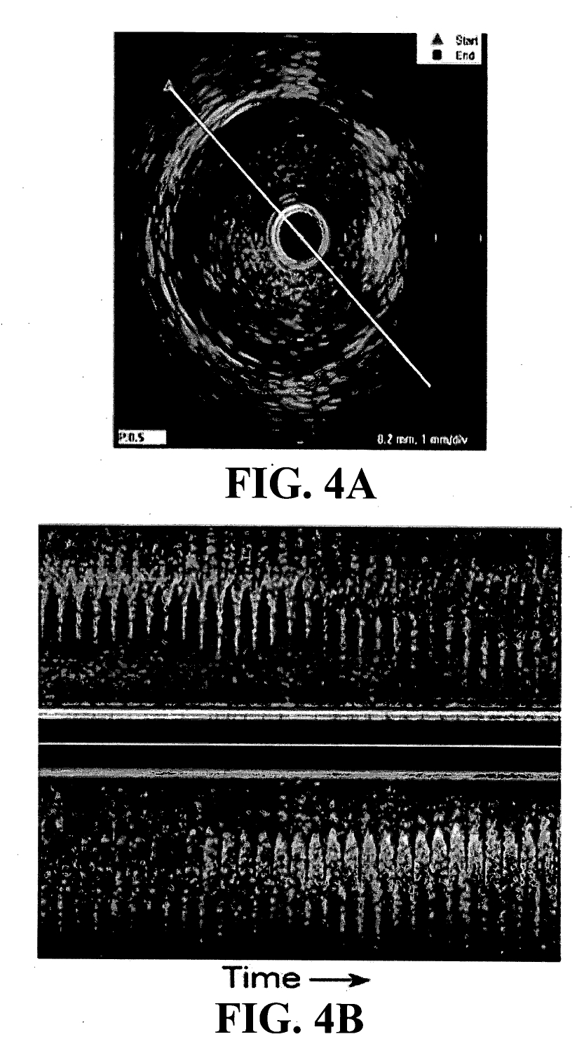

Gating methods based on ECG are effective for two reasons. If data are always collected when the heart is in a similar pose, the data will be more consistent. Second, if the data are collected at a point in time when the heart is relatively motionless, motion-blur artifacts will be reduced. In IVUS, gating is used to reduce motion artifacts otherwise visible in the volumetric vessel images reconstructed from pullback sequences or recordings [1] [4]. Without gating, the long (time) axis of these volumes present sawtooth-like artifacts which confound data analysis. For S-C IVUS studies, gating is used as a preprocessing step to alleviate motion before more detailed analyses of the vessel are performed [5] [7]. FIGS. 4A&B illustrate these sequences further.

The first question that arises in the context of gating is whether the ECG signal should be used at all. One practical difficulty with ECG-gating is that of acquiring the signal and guaranteeing synchronization with the captured images. A more difficult conceptual problem and usually not obvious is that of choosing the most effective R-R fraction at which to gate in order to obtain maximal inter-frame stability. In other modalities, the selection of the appropriate R-R fraction may involve a function of(1) the site being imaged (i.e., which artery), (2) the heart rate of the subject, and (3) the modality in question [8], [9]. There is little reason to believe similar principles do not apply to IVUS imaging. Regardless, for most studies the 0% point (i.e., the R-wave itself) is usually chosen. While this point is not necessarily optimal, selecting a fraction other than this can be subject to decreased performance in the presence of certain heart rate variations, as interpolation from the R-wave landmarks is then needed [10].

To circumvent some of these ECG-related problems and allow gating to be performed on sequences for which ECG signals are not available, methods have been developed which attempt to derive ECG-like signals directly from the sequences data. It may be difficult to reliably locate suitable landmarks in these signals, however, and they often gate at an arbitrary (and unknown) fraction of the R-R interval [11]. Due to frequency-estimation issues, they may also be inflexible to variations in the heart rate of the subject during recording. Given a segmentation of each frame, it is possible to overcome many of these problems [12]; unfortunately, reliable fully-automated IVUS segmentation tools do not currently exist. An image-based gating method has been proposed which aims to locate the frames captured nearest in time to the R-waves, but few details are provided about its operation [13].

One issue apparently ignored so far is that, in some cases, it may not be desirable to retain only one frame per cardiac cycle. In a simple case where the heart rate is 60 beats/min and the IVUS frame rate is 30 frames/s, cardiac gating will eliminate a significant fraction (29 out of 30) of the data frames in the sequence. For some applications, such as functional imaging, this data reduction may be undesirable. Thus, there would be an advantage for method that relaxes the one-frame-per-cycle rule, while still making reasonable choices about clustering “similar” frames in the sequence into related ensembles. This is the motivation behind the S-C gating scheme described herein. As far as the inventors are aware, similar methodologies have not been proposed in the medical imaging community; however, our method could be considered a form of video event detection.

Section III—Materials & Methods

A. IVUS Sequences

Pullback sequences were obtained in vivo in the coronary arteries of normal swine using a 40 MHz IVUS system. The pullback sequences were obtained at a pull rate of 0.5 mm/s and at a frame rate of 30 frames/s. Each recorded sequence contained ˜2000 frames, providing images from vessel segments ˜30 mm in length.

Stationary-catheter sequences were obtained in vivo in human patients with coronary artery disease using a 20 MHz IVUS and atherosclerotic swine using a 40 MHz IVUS. Recording occurred over a matter of minutes, approximately halfway through which an intra-coronary bolus injection of a micro-bubble contrast agent was made proximally to the imaging catheter [6]. Passage of the contrast agent through the lumen leads to a brief washout of the IVUS image, as the bubbles are echo-opaque in high concentrations.

B. Dissimilarity Matrix Construction

For both pullback and S-C sequences, the following methods operate on dissimilarity matrices constructed from pair-wise comparisons of frames in the sequence. Specifically, given an n-frame sequence, a symmetric, n×n proximity matrix D is constructed, where each entry d_{i,j} represents a dissimilarity value between frames i and j. Almost any registration metric may be used to derive this dissimilarity. In this embodiment of the method, the inventors used normalized cross-correlation (NCC), though in principle an ultrasound-specific metric such as CD₂ [14] or CD_{2bis} [15] could also be employed. While NCC returns values on the interval [−1,+1], the inventors clamp these values to the interval [0,+1] and subtract the resulting value from one. This results in a matrix where (1) the main diagonal is everywhere zero and (2) all other entries are non-negative, with frame pairs which differ more in appearance representing a larger positive value. When other registration metrics are used, these two properties can be imposed typically by data remapping.

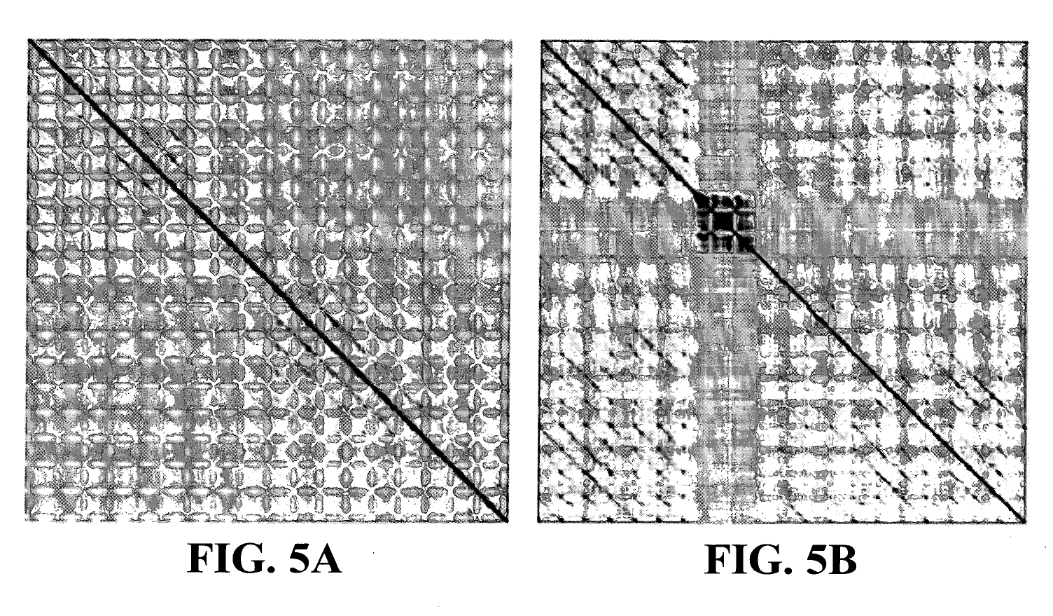

Matrices from a pullback and a S-C sequence are depicted graphically in FIGS. 5A&B, respectively. Both types of matrices exhibit a periodic structure, as the changes in IVUS image appearance due to the beating heart are far more rapid than any other change that will occur during recording. For illustration purposes, these matrices will be shown in full, however, in Section III-D the inventors will describe methods to avoid the computational cost of full matrix construction.

C. Gating

1) Pullback Sequence Gating

When D is derived from a pullback sequence, the inventors seek to extract a series of frames such that (1) one frame is picked per cardiac cycle, (2) the frames are picked at a point in the cycle, when the heart is relatively motionless, and (3) all the frames are at roughly the same fraction of the R-R cycle (i.e., so that in each frame the heart is in a similar pose).

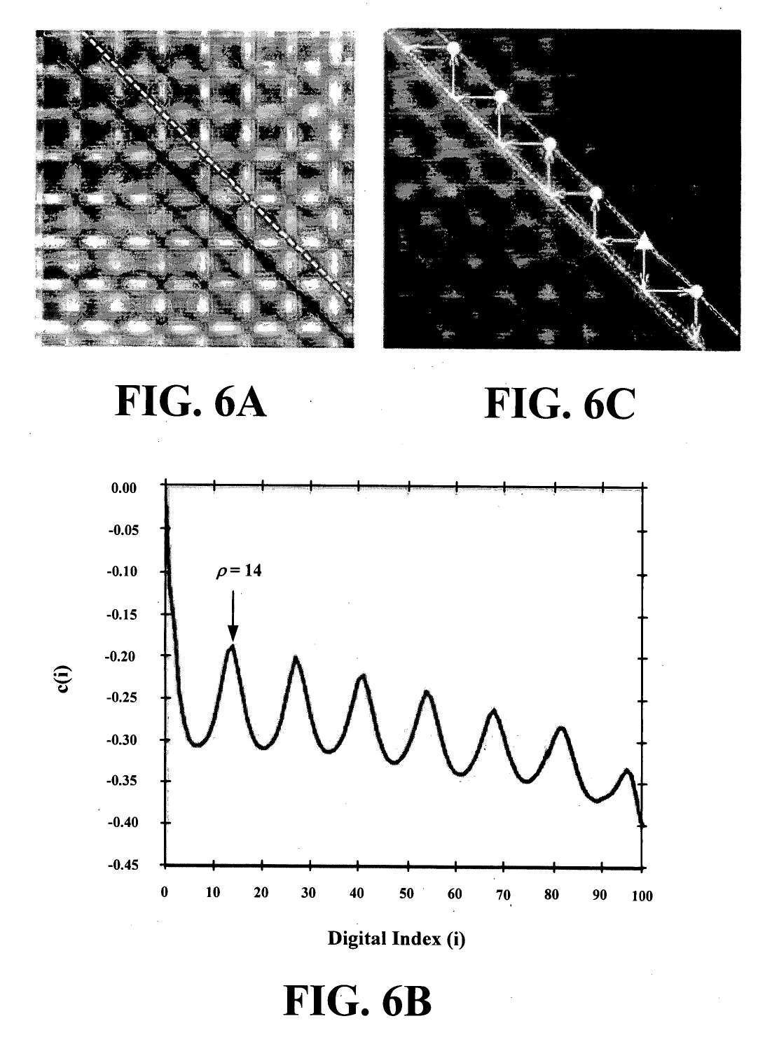

To begin, a rough estimate of the heart rate over the entire recording is obtained using the function

$$c ( i ) = - \frac{1}{n - i} {\sum }_{j = 1}^{n - i} {d}_{i + 1 , j} \qquad ( 2 )$$

where i ranges from 0 to n_{—}1 (i.e., indexing the i^{th} diagonal). Next, the index p of the first peak is found from the left in this signal (see FIG. 6B). Due to the amount of redundancy present in the matrix, this point is usually unambiguous. The value p represents the average length, in frames, of the cardiac cycle over the complete recording. To see why this is the case, note that if p is the known length of the cardiac cycle (in frames), then for a given frame i, there will be a diagonal-parallel valley around entry d_{i,i+p}, indicating that the heart has achieved the same pose at frame i+p as at frame i. The function c will exhibit peaks at these off-diagonal valleys.

While at this point, the inventors have an estimate of the overall heart rate, the inventors do not know, if given a specific frame i, the time offset from i returns the heart to the same pose. If this offset were exactly p for all frames, then the inventors would expect that d_{i,i+p}<d_{i,i+p-1} and d_{i,i+p}<d_{i,i+p+1}. However, perturbations arise in the data due to changing heart rate and to how the IVUS frame rate imposes a discretization on the real-valued heart rate in every cycle. To find a more accurate offset from each frame, the method traces a path v along the off-diagonal valley, which represents the cardiac cycle length locally at each frame (see FIG. 6A). This is accomplished through a dynamic programming step that begins at d_{1,p} and traces down and to the right. That is, each step proceeds either one entry downward, one entry to the right, or one entry downward and to the right, diagonally downward. In practice, this tracing step operates only on a narrow band around the p^{th} diagonal to tracing routine from seeking the main diagonal. This “band width” restriction can be set to a fraction of p so that it adjusts to the heart rate of the subject. Tracing terminates when the path v exits D near its lower-right corner, globally minimizing the sum of all matrix entries through which the path v traverses. A second tracing step can be performed from the end-point toward the upper-left so as to make this procedure invariant to the starting point, d_{1,p}.

It remains to determine a set of frames, each captured at the same point in the cardiac cycle, which is associated with a point in phase when the heart is maximally motionless. The inventors note that if the path traced earlier passes through a point (i,j), this indicates that the heart obtains the same position in frame j as it did in frame i. In addition, if i and j are captured when the heart is moving slowly, the valley around (i,j) will be more pronounced. There will also be low-dissimilarity structures in the matrix D that are perpendicular to the main diagonal at these points. To accentuate both of these features, the methods constructs a matched filter in the form of an X-shaped, inverted Gaussian kernel given below

$${G}_{σ} ( x , y ) = \{ \begin{matrix}- exp ( - \frac{{x}^{2} + {y}^{2}}{2 {σ}^{2}} ) & if \vert x \vert = \vert y \vert \\ 0 & otherwise\end{matrix} \qquad ( 3 )$$

where σ=[p/3]. The inventors now define \hat{D} [symbol] G_{σ}, where [symbol] denotes convolution. The matrix \hat{D} exhibits maxima in areas where a frame pair is associated by both high similarity and low motion.

To begin the method's final step, a single phase-associated frame pair is selected which confidently represents a maximally-stable point in the cardiac cycle. A trace through \hat{D} along v to is then used to find a global maximum, (s₀, t₀). This starting point and v is used to proceed step-wise upward and downward through \hat{D}, collecting the frames which will comprise a gated sequence (see FIG. 6C). The downward step sequence is as follows.
1. Let i←0
2. The point on the diagonal below (sᵢ, tᵢ) is (tᵢ, tᵢ). Locate the column j where v intersects row tᵢ. If this does not exist, then the end of the sequence has been reached and the step can be stop. Otherwise, let (sᵢ₊₁, tᵢ₊₁)=(t_{i,j}).
3. Following a simple gradient ascent, adjust the position of (sᵢ₊₁, tᵢ₊₁) to a local maximum of \hat{D}. This again helps account for heart rate/sampling variations.
4 Let i←i+1
5: Repeat to Step 2-4 until the step-wise process in completed.
   Stepping upward proceeds analogously. Assuming that after these step-wise processes the series of off-diagonal points that are collected are ordered chronologically as (u₀, v₀), (u₁, v₁), . . . , (uₘ, vₘ), then the frame numbers in the gated sequence are indicated by {u₀, v₀, u₁, v₁, . . . , uₘ, vₘ}.

2) Stationary-Catheter Sequence Gating

When D is derived from a S-C sequence, the method is designed to divide the n-frame sequence into a series of k ensembles, where each ensemble contains a group of “similar” frames. Next, assume, as the catheter is not moved during these recordings, that frames which appear to be similar do in fact represent a similar pose of the artery relative to the IVUS imaging catheter. However, anomalies such as unintentional catheter motion (nudging or slippage) can also be detected during this process.

The first step in this process is to convert the n-frame sequence represented by D into a Euclidean frame-similarity space in which each frame is represented by a single point and groups of similar frames are located nearby spatially. This is accomplished with multidimensional scaling (MDS): a technique for transforming pair-wise distance information, here values in D, to a point cloud in which the original inter-point distances are approximated. For consistency with prior literature on MDS, the notation of Seber [16] is used for the majority of this section. Vectors are columnar unless otherwise noted.

To create the frame-similarity space, let A be the matrix where

$${a}_{i , j} = - \frac{1}{2} {d}_{i , j}^{2} \qquad ( 4 )$$

and let Cn be the n×n centering matrix,

$${C}_{n} = {I}_{n} - \frac{1}{n} {1}_{n} {1}_{n}^{T} \qquad ( 5 )$$

where I is the identity, 1 is a vector of unit entries, and ^{T} indicates transpose. Now, let
B=CₙACₙ (6)
which is the double-centered A. Letting λ₁≦λ₂≦ . . . ≦λₙ and v₁, . . . , vₙ be the eigenvalues and associated eigenvectors of B, and q the number of positive eigenvalues, a matrix Y is formed
Y=(\sqrt{λ₁}v₁, \sqrt{λ₂}v₂, . . . , \sqrt{λ_{q}}v_{q}) (7)

Each row of Y specifies the coordinates of a point in the q-dimensional frame-similarity space (i.e., the i^{th} row corresponds to the i^{th} frame in the sequence). As mentioned, the Euclidean inter-point distances in this space are necessarily an approximation of the distances in the non-Euclidean matrix D. This is not a problem for the method of this invention; in fact, the dimensions of the space described by Y can be further reduced to fewer than q if needed to make subsequent computations less expensive. Essentially, this consists of removing one or more of the rightmost columns of Y according to the magnitude of the associated eigenvectors (an almost identical procedure to that used in principal component analysis). For visualization purposes, only the first two or three dimensions may be plotted.

Given the set of n points in the q-dimensional frame-similarity space defined by Y, it remains to cluster these points into meaningful ensembles. These ensembles, in a general video-analysis sense, could be said to represent “events,” but in present context, these ensembles typically represent common orientations of the catheter with respect to the vessel wall. Hence, some clusters represent the stabilized frame sets sought, eliminating the expected periodic motions of the heart, while outlying points and clusters may indicate the occurrence of unusual events such as the catheter being nudged.

Almost any spatial clustering algorithm maybe employed on the space at this point; common choices include hierarchical clustering and k-means. It is noted that while spectral clustering [18], [19] may seem an obvious choice when working with proximity matrices as it would allow avoid avoidance of the MDS methodology entirely, its strength is in clustering connected components. Here, proximity-based grouping is desired. Note that for clustering purposes, it is safe to make the assumption that the derived space is isotropic; that is, a hyper-sphere at a particular point in this space will contain an ensemble of frames which are similar according to a threshold determined by its radius. For this reason, methods such as Gaussian mixture modeling are avoided, which produces anisotropic cluster boundaries. Instead, here randomly-initialized k-means with multiple runs to converge to a lowest-error clustering are used. A human operator selects k from a visualization of the clusterings associated with several different k values, the goal being to locate an ensemble which includes a number of frames which is reasonable for a particular analysis (e.g., ˜50 pre-contrast and ˜100 post-contrast frames). Note that a large number of groups, k, implies that each group will be smaller but more similar (stable) than otherwise. Therefore, a balance is struck between the length and stability requirements of the gated sequences. The inventors have found that manual selection of the parameter k is a convenient way of making this decision, though many other clustering methods with greater or lesser human interaction could be devised.

D. Computational Considerations

1) Pullback Gating

The primary source of complexity in the methodology described herein is the construction of the dissimilarity matrix, D; this is an O(n²) operation in the number of frames as n(n−1)/2 pair-wise comparisons must be performed. However, note that the method actually only operates on a narrow band of D. The width of this band is dependent on the length of the cardiac cycle as well as the IVUS frame rate. Hence, if letting p be an estimate of the minimum heart rate (in beats/min) expected in any subject and letting φ be the frame rate (in frames/s), then comparing a frame to only its

$$2 [ \frac{60 ϕ}{ρ} ]$$

successors reduces matrix formation to O(n). The multiplication by 2 is to provide padding in the convolution to find \hat{D}.

2) Stationary-Catheter Gating

While the O(n³) cost associated with the eigenvector calculation required by MDS is often considered to be its bottleneck [20], this is not necessarily the case in our application. Some registration metrics may be expensive enough that, similarly to the pullback-gating problem, the actual burden comes from constructing D. However, unlike spectral-clustering approaches such as normalized cuts [18], classical MDS does not allow us to sparsify our matrix simply by ignoring (e.g., setting to zero) some of its entries.

There are two ways to address this problem. The first is to use a more efficient similarity metric; for instance, multiresolution histogram [21] [24] have been successfully used. This method associates with each image a short feature vector consisting of a series of concatenated cross-resolution difference histograms. Instead of comparing image pairs directly, the L₁ distance between a pair of these feature vectors is used.

Of course, choosing a comparison metric based only on its computational expense is not an option if a specialized metric is required for a particular task; it would be preferable to instead limit the amount of work required to fill D. The inventors therefore consider sparse dissimilarity matrices, and note that non-metric multidimensional scaling (NMDS) approaches have been developed which allow the creation of a similarity space from incomplete information [25] [27]. For the present application, NMDS allows banded or other sparse dissimilarity matrices to be employed, reducing the time complexity of forming D from quadratic to near-linear. The inventors have shown that using a banded matrix that eliminates 50-60% of the matrix entries allows Y to be constructed with an accuracy comparable to a full-matrix MDS solution. Other types of matrix (e.g., symmetric random matrices) can also be used to provide better results with greater sparsity.

Section IV—Results

A. Pullback Sequence Gating

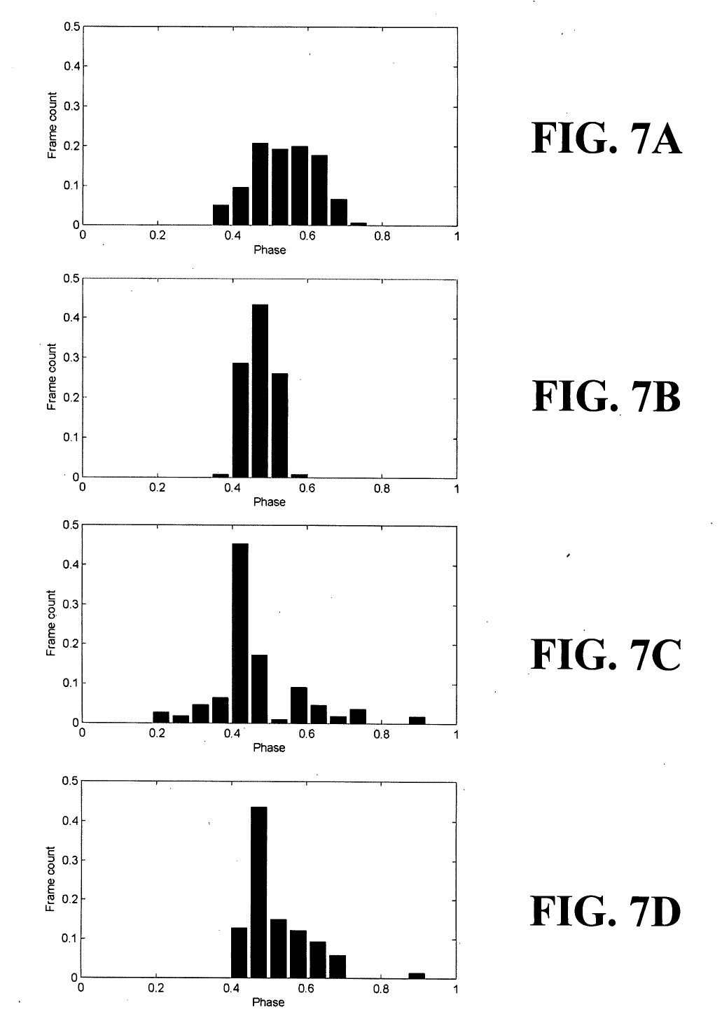

To compare the non-ECG gating method of this invention with other methods, four IVUS pullbacks along with synchronized ECG signals were recorded in vivo in healthy swine. Properties of the frames picked by the method of this invention were then compared against those picked by an ECG based method. These results are summarized in Table I, where n is the count of frames in the sequence, 6 is its physical length, n_{ecg} and n_{alg} are the counts of frames gated by ECG and the gating method of this invention, and μₚₕₐₛₑ and σₚₕₐₛₑ are the mean and standard deviation of the fraction of the R-R cycle of the selected frames from the methods. In FIGS. 7A-D, the relationship between the picked frames from the gating method of this invention and an ECG-based gating method is illustrated in more detail. The discrepancy between the number of frames picked by the two methods and the isolated histogram outliers are due to the phase offset between the methods and the boundary conditions of the sequence, and are expected. The “spread” of the histograms is also expected, as the 970 Hz ECG signals must be re-sampled onto the 30 Hz frame sequences, leading to quantization effects. In general, though, lower σₚₕₐₛₑ values indicate better reproduction of ECG behavior. The significance of the μₚₕₐₛₑ values and other issues will be discussed further in Section V.

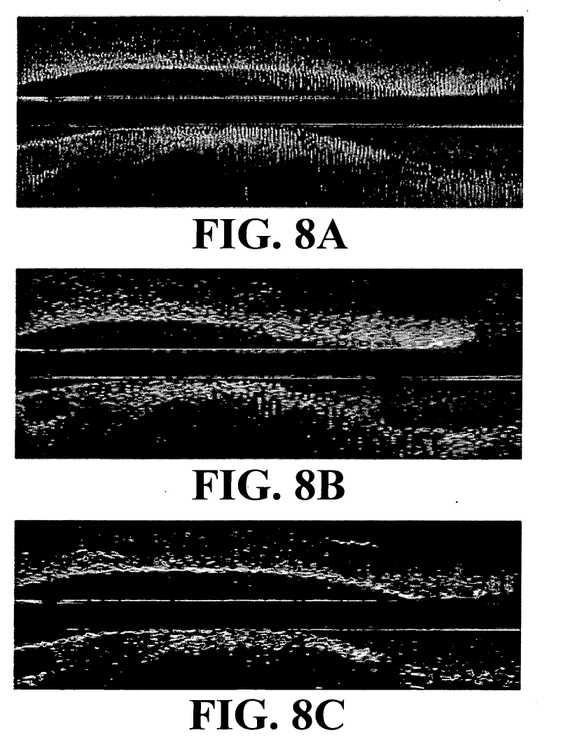

As our ultimate goal is the reconstruction of pullback volumes, we visually compare these gating methods in FIGS. 8A-C.

B. Stationary-Catheter Sequence Gating

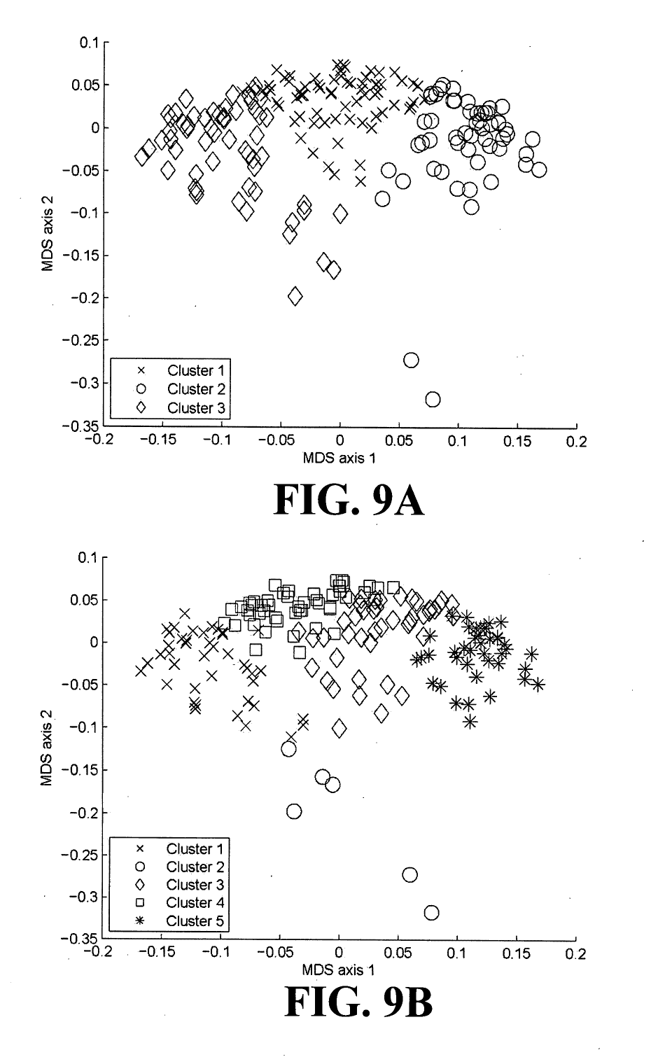

Clusterings of a frame-similarity space for k=3 and k=5 are shown in FIGS. 9A-B. These clusters represent stabilized frame sequences, which could be compiled into their own video sequences before subsequent analysis. Taking a closer look at the similarity space in FIG. 9B, a cluster consisting of several outliers is observed. These outliers represent frames captured during contrast injection and could be eliminated if necessary. The method can also be used to extract the “most typical” frame associated with each cluster by picking the frames nearest the cluster centroids. These frames can be used, for instance, to provide a human observer with a visual summary of the events occurring in the sequence.

TABLE I
Comparison of Four Pullback Cases

| # | n | δ | n_{ecg} | n_{alg} | μₚₕₐₛₑ | σₚₕₐₛₑ |
|---|---|---|---|---|---|---|
| 1 | 1828 | 30.5 mm | 135 | 135 | 54% | 8.1% |
| 2 | 1945 | 32.4 mm | 116 | 115 | 47% | 4.4% |
| 3 | 1774 | 29.6 mm | 109 | 110 | 47% | 12.0% |
| 4 | 2283 | 38.0 mm | 140 | 140 | 53% | 7.8% |

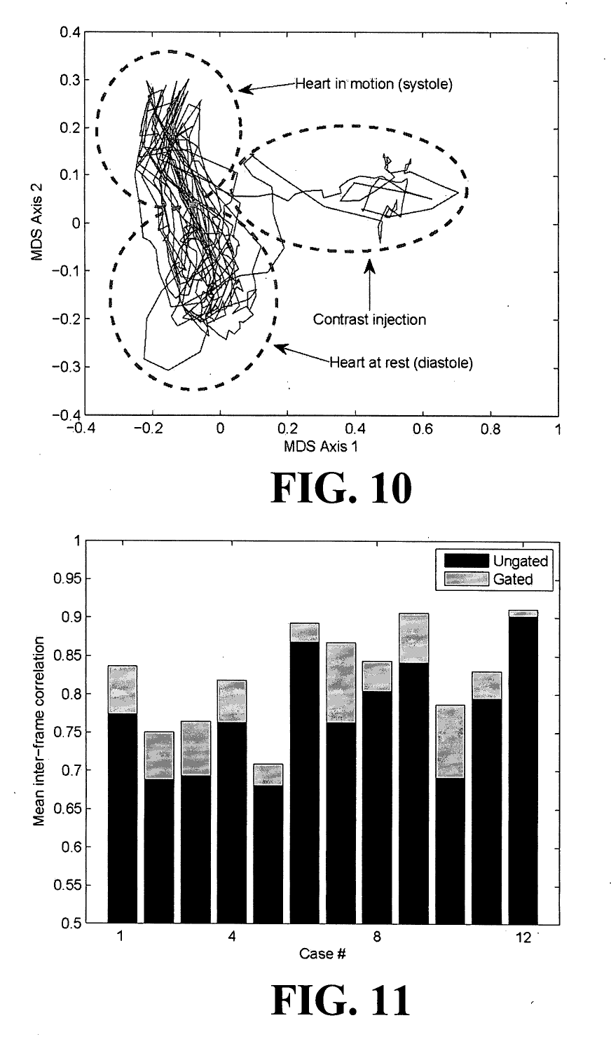

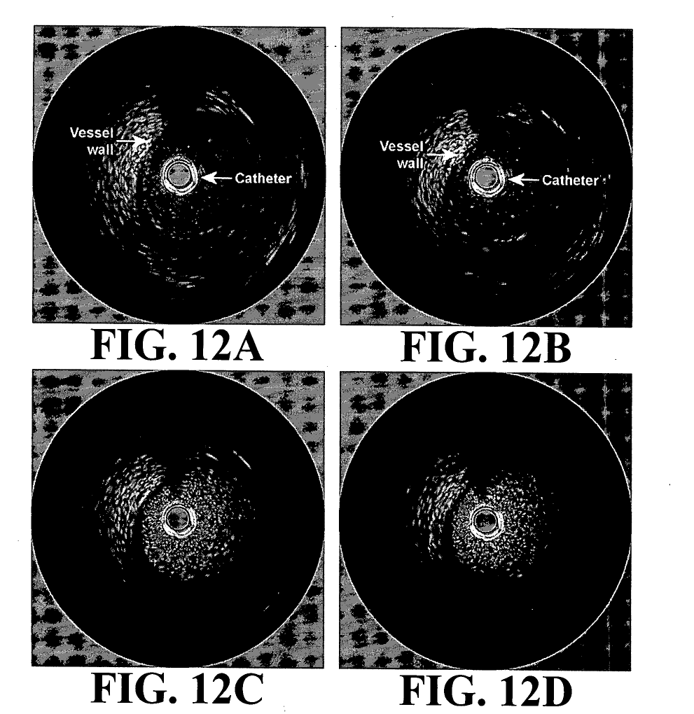

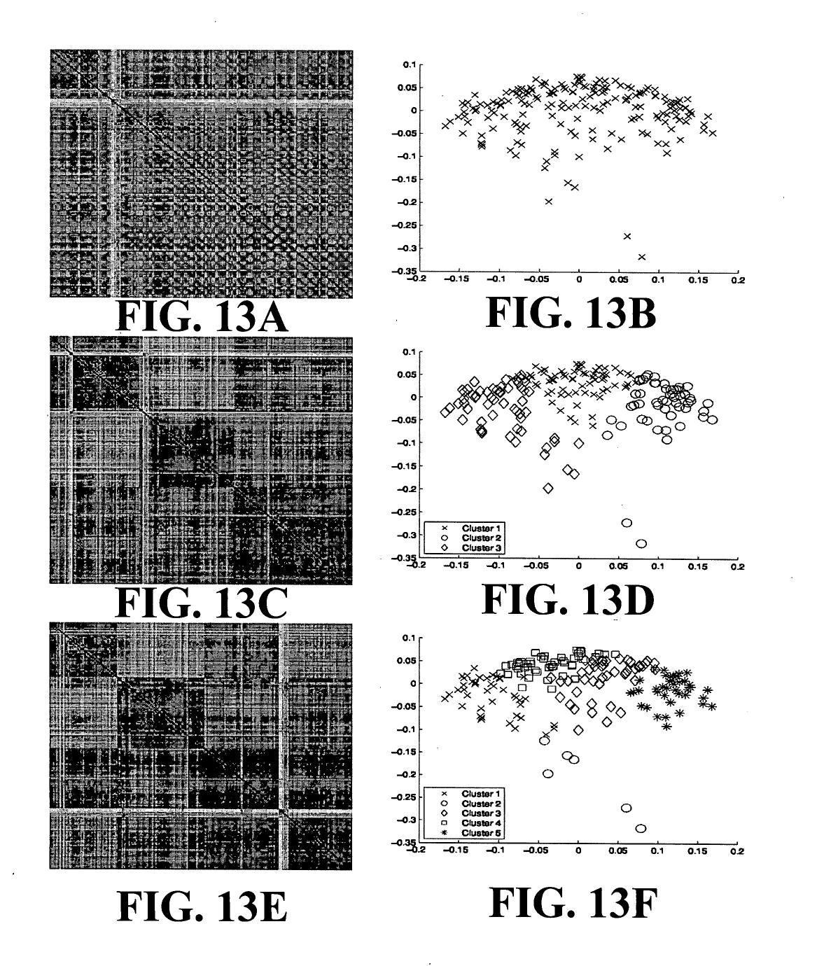

Other high-level interpretations of these spaces are possible, e.g., in FIG. 10. Note that while frame-similarity spaces are capable of being analyzed in a high-level manner, in practice this is not necessary as our only goal is to group clusters of related frames. Specifically, a human operator picks the cluster which best fulfills the needs of a particular study. In FIG. 11, pair-wise cross-correlation of the grey levels of all frames in a sequence before and after clustering are performed, and then take the mean of these cross-correlations. High means indicate a better clustering. Here, the operator has selected clusters containing ˜100 frames in each case. Examples of gating results are given in FIGS. 12A-D and FIGS. 13A-F.

Section V—Conclusion

We have described image-based frame gating methods for stationary-catheter and pullback IVUS sequences. These methods rely on the analysis of dissimilarity matrices derived from pairwise frame comparisons.

For pullback gating, we note that the algorithm's R-R fraction selection varies slightly by subject (47-54%), as we would expect from prior research. Such variability could not be obtained by blind ECG triggering based on a fixed R-R fraction. While we have chosen to pick the most visually-stable points in the sequence as our gating points, these tended to be at roughly the same R-R fraction (˜50%). This being the case, truer ECG emulation could be accomplished by temporally shifting the algorithm-selected frames appropriately. However, as previous studies have hinted (Section II of this portion of the specification), ECG may not be a reliable standard to aspire to.

Our second gating system operates on the stationary-catheter sequences employed in IVUS perfusion imaging. Our implementation requires minimal manual guidance, consisting of selecting a cluster from among those generated by several iterations of k-means. However, given application-specific criteria (e.g., a minimum cluster size), it would not be difficult to completely automate this process.

We have not tested either method on pathological cases (e.g., subjects with irregular heartbeat) and have not modeled how these would affect performance. However, we expect such anomalies would have lesser impact on the S-C gating method than the pullback method, which has stricter gating requirements (i.e., exactly one frame per cycle). Future work will involve further validation and refinement to account for such special cases.

A more complete discussion of the topics presented in this portion of the specification may be found in [17].

### REFERENCES CITED IN IMAGE-BASED GATING

The following references were cited in the Image-based Gating of Pullback and Stationary-catheter Sequences in Intravascular Ultrasound.
[1] R. M. Cothren, R. Shekhar, E. M. Tuzcu, S. E. Nissen, J. F. Corhill, and D. G. Vince, “Three-dimensional reconstruction of the coronary artery wall by image fusion of intravascular ultrasound and bi-plane angiography.” Int J Cardiac Imaging, vol. 16, no. 2, pp. 69-85, 2000.
[2] S. M. O'Malley, S. G. Carlier, M. Naghavi, and I. A. Kakadiaris, “Imagebased frame gating of IVUS pullbacks: A surrogate for ECG,” in IEEE Acoustics, Speech, Sig Proc, April 2007.
[3] R. Shekhar, R. M. Cothren, D. G. Vince, S. Chandra, J. D. Thomas, and J. F. Cornhill, “Three-dimensional segmentation of luminal and adventitial borders in serial intravascular ultrasound images,” Comput Med Imaging Graph, vol. 23, no. 6, pp. 299-309, 1999.
[4] M. Sonka, W. Liang, X. Zhang, S. DeJong, S. M. Collins, and C. R. McKay, “Three-dimensional automated segmentation of coronary wall and plaque from intravascular ultrasound pullback sequences,” in Computers in Cardiology, 1995, pp. 637-640.
[5] S. Carlier, I. A. Kakadiaris, N. Dib, M. Vavuranakis, S. M. O'Malley, K. Gul, C. J. Hartley, R. W. Metcalfe, R. Mehran, C. Stefanadis, E. Falk, G. Stone, M. Leon, and M. Naghavi, “Vasa vasorum imaging: A new window to the clinical detection of vulnerable atherosclerotic plaques,” Curr Atheroscler Rep, vol. 7, no. 2, pp. 164-169, March 2005.
[6] S. M. O'Malley, M. Vavuranakis, M. Naghavi, and I. A. Kakadiaris, “Intravascular ultrasound-based imaging of vasa vasorum for the detection of vulnerable atherosclerotic plaque,” in Med Image Comput Comput Assist Interv, vol. 1, October 2005, pp. 343-351.
[7] S. M. O'Malley, M. Naghavi, and I. A. Kakadiaris, “Image-based frame gating for stationary-catheter IVUS sequences,” in Workshop on Computer Vision for Intravascular & Intracardiac Imaging, Copenhagen, Denmark, October 2006.

[8] S. Leschka, L. Husmann, L. M. Desbiolles, O. Gaemperli, T. Schepis, P. Koepfli, T. Boehm, B. Marincek, P. A. Kaufmann, and H. Alkadhi, “Optimal image reconstruction intervals for non-invasive coronary angiography with 64-slice CT,” Eur Radiol, vol. 16, no. 9, pp. 1964-1972, September 2006.
[9] B. Lu, S.-S. Mao, N. Zhuang, H. Bakhsheshi, H. Yamamoto, J. Takasu, S. C. K. Liu, and M. J. Budoff, “Coronary artery motion during the cardiac cycle and optimal ECG triggering for coronary artery imaging,” Invest Radiol, vol. 36, no. 5, pp. 250-256, 2001.
[10] N. Bruining, C. von Birgelen, P. J. de Feyter, J. Ligthart, W. Li, P. W. Serruys, and J. R. T. C. Roelandt, “ECG-gated versus nongated three-dimensional intracoronary ultrasound analysis: Implications for volumetric measurements,” Cathet Cardiovasc Diagn, vol. 43, no. 3, pp. 254 260, March 1998.
[11] H. Zhu, K. D. Oakeson, and M. H. Friedman, “Retrieval of cardiac phase from IVUS sequences,” in SPIE Medical Imaging: Ultrasonic Imaging and Signal Processing, vol. 5035, February 2003, pp. 135-146.
[12] S. K. Nadkarni, D. Boughner, and A. Fenster, “Image-based cardiac gating for three-dimensional intravascular ultrasound imaging,” Ultrasound Med Biol, vol. 31, no. 1, pp. 53-63, January 2005.
[13] S. A. de Winter, R. Hamers, M. Degertekin, K. Tanabe, P. A. Lemos, P. W. Sermuys, J. R. T. C. Roelandt, and N. Bruining, “Retrospective image-based gating of intracoronary ultrasound images for improved quantitative analysis: The Intelligate method,” Catheter Cardio Inte, vol. 61, no. 1, pp. 84-94, January 2004.
[14] B. Cohen and I. Dinstein, “New maximum likelihood motion estimation schemes for noisy ultrasound images,” Pattern Recogn, vol. 35, no. 2, pp. 455-463, February 2002.
[15] D. Boukerroui, J. A. Noble, and M. Brady, “Velocity estimation in ultrasound images: A block matching approach,” in Inf Process Med Imaging, 2003, pp. 586 598.
[16] G. Seber, Multivariate Observations. Wiley, 1984, ch. 5.5: Multidimensional scaling.
[17] S. M. O'Malley, “Computational methods for contrast-enhanced intravascular ultrasound sequence analysis,” Ph.D. dissertation, University of Houston, May 2007.
[18] J. Shi and J. Malik, “Normalized cuts and image segmentation,” IEEE T Pattern Anal, vol. 22, no. 8, pp. 888-905, August 2000.
[19] Y. Weiss, “Segmentation using eigenvectors: a unifying view,” in IEEE Intl Conf Comp Vis, 1999, pp. 975-982.
[20] T. Yang, J. Liu, L. McMillan, and W. Wang, “A fast approximation to multidimensional scaling,” in Workshop on Computation Intensive Methods for Computer Vision, May 2006.
[21] E. Hadjidemetriou, M. D. Grossberg, and S. K. Nayar, “Multiresolution histograms and their use for recognition,” IEEE T Pattern Anal, vol. 26, no. 7, pp. 831-847, July 2004.
[22] P. Jain and S. N. Merchant, “Wavelet based multiresolution histogram for fast image retrieval,” in IEEE Intl Region 10 Conf, vol. 2, October 2003, pp. 581-585.
[23] O. D. Robles, P. Toharia, A. Rodriguez, and L. Pastor, “Automatic video cut detection using adaptive thresholds,” in IASTED Vis, Imaging, and Image Proc, September 2004, pp. 517-522.
[24] B. C. Song, M. J. Kim, and J. B. Ra, “A fast multiresolution feature matching algorithm for exhaustive search in large image databases,” IEEE T Circ Syst Vid, vol. 11, no. 5, pp. 673-678, May 2001.
[25] J. B. Kruskal, “Multidimensional scaling by optimizing goodness of fit to a nonmetric hypothesis,” Psychometrika, vol. 29, pp. 127, 1964.
[26] I. Spence and D. W. Domoney, “Single subject incomplete designs for nonmetric multidimensional scaling,” Psychometrika, vol. 39, no. 4, pp. 469-490, December 1974.
[27] L. Tsogo, M. H. Masson, and A. Bardot, “Multidimensional scaling methods for many-object sets: A review,” Multivar Behav Res, vol. 35, no. 3, pp. 307-319, 2000.

### E. Difference Imaging for Contrast Detection

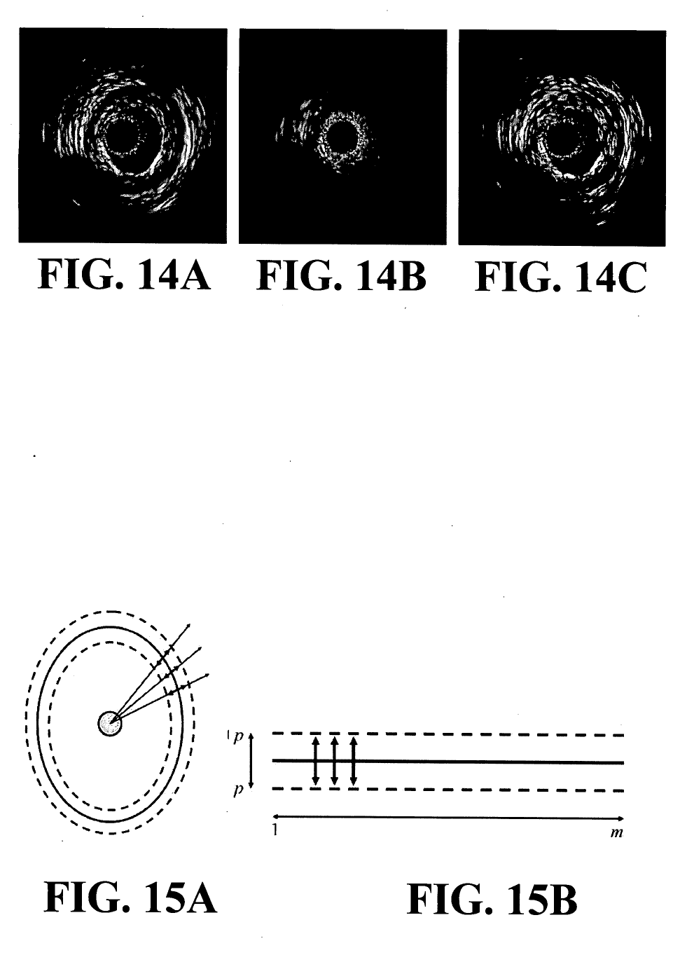

The present invention also relates to a method for difference imaging analysis, where the method is adapted to detect regions of contrast perfusion into a vessel wall as shown in FIGS. 14A-C. The steps performed to use difference imaging-based change detection to discover regions of contrast perfusion into the vessel wall are as follows:
1. positioning a catheter at a maximally-stenotic point of a suspect plaque or a region of interest (ROI);
2. imaging the ROI for a first period of time, generally on the order of 30 seconds, while holding the catheter steadily in place;
3. injecting a bolus dose of a contrast agent or contrast effect intra-coronarily, proximate the imaging catheter;
4. imaging the ROI for a second period of time, generally on the order of 30 seconds, again while holding the catheter steadily in place;
5. image-based frame gating the data to decimate a number of frames in the sequence of image frame collected in steps 2 and 4, providing a stabilized gated sequence with fewer frames than the original;
6. outlining an inner and outer contours of the ROI in the first frame of the gated sequence—performed either by an operator or an outlining routine;
7. propagating the ground-truth contours to the remaining frames in the sequence, in order to provide a segmentation of each frame;
8. extracting a region between the contours into a rectangular raster in each frame, providing a stabilized space in which inter-frame comparisons of the ROI are to be performed;
9. averaging a pre-injection ROI images to obtain a pre-injection baseline of the non-contrast ROI;
10. subtracting the averaged baseline ROI pixel-wise from the pre- and post-injection ROI images to detect differences between the baseline appearance and the pre- and post-contrast appearance, where the pre-injection frames will rarely exhibit any changes as the pre-injection baseline is derived from them; and
11. mapping the difference-imaged ROIs back into the original IVUS space for visualization and quantification of the changes which occurred due to contrast perfusion.

The above method is explained in greater detail below in Section 1.1.1 through Section 1.2.3

Section 1.1.1 Contour Tracking

Given a frame-gated sequence, we assume that axial catheter motion has been essentially eliminated. The residual motion artifacts are generally much milder, but are still significant. In order to eliminate these, it is necessary to introduce either a segmentation method capable of indicating what these transformations are over time. In principle, any segmentation method could be used for this purpose, as a segmentation of the luminal and medial borders would provide information about both the rigid transformations due to relative catheter/vessel motion as well as the elastic deformations of the vessel wall. However, during the course of the work undertaken in this thesis, it became clear that these general segmentation algorithms were unsuitable when applied to highly diverse types of data. For instance, the earliest methods for luminal segmentation assume that the lumen is relatively echo free, as is typical for 20 MHz catheters. When higher-frequency catheters (30-40 MHz) came into wider use, their increased blood echogenicity reduced the contrast at the luminal edge, leading to segmentation methods which instead assume the presence of significant luminal speckle. Frequency differences aside, many of these methods are also incapable of providing a reasonable segmentation in the presence of acoustic shadowing and atypical image features such as adjacent vessels. Those which segment the media-adventitia border may also assume the visible presence of the media, which is not always the case.

In addition to these normal inter-sequence variations in image quality due to the recording site and the imaging hardware and software, the present invention also seeks to analyze recordings made in vivo in humans (20 and 40 MHz) and in swine (40 MHz). While anatomically similar, the swine data typically suffer from greater motion artifacts, a more elastic vessel wall, and more homogeneous plaques.

Taking all these differences into consideration, it is clearly impractical to develop and tune segmentation techniques capable of handling every combination of expected variations in image properties. As such, we instead focus on contour tracking as opposed to segmentation, though our method draws techniques from both areas. By contour tracking, we mean that we segment an image based on an example contour drawn by a human operator on a related image (i.e., an image from the same sequence). Typically, the contour will be drawn in the first frame of the sequence and this knowledge will be used to segment the same contour on all subsequent frames. Essentially, this could be considered segmentation with a strong prior. The method of this invention also differs from traditional contour tracking in that we do not propagate the contour; instead, knowledge acquired from the prior segmentation is used to segment all other frames independently, not sequentially. While contour tracking is a well-studied area of computer vision [3], it is not immediately applicable here. This is for several reasons. First, there is a high probability of error propagation in our application due to the contrast injection, which wipes out significant portions of the image one or more times over the course of the sequence. Second, shadowing effects frequently cause portions of the image to lack any trackable features entirely. Third, IVUS images are highly cluttered due to the appearance and disappearance of adjacent features around the contour of interest. Finally, classical contour tracking methods would still require an initial contour to propagate; they do not address the segmentation problem.

Of course, the disadvantage of our method is that a human operator must provide a contour of interest. The advantage is that it otherwise automatically tunes itself to the sequence at hand, and is generally capable of operating on a wide variety of sequences without any sequence-specific adjustment. In addition, we may segment arbitrary boundaries of interest, not necessarily anatomically-meaningful boundaries or even those associated with visible image features. This is a distinct benefit if a non-standard ROI needs to be analyzed or in the presence of severe artifacts due to shadowing or the guide wire; if the human operator provides a reasonable contour through a region of poor image quality, our tracking method will generally mimic this. Otherwise, when relevant image features are available, these are exploited.

Our method follows a two-step approach: a rough, rigid alignment step followed by an elastic refinement step.

Definitions and Conventions

Following the standard convention in the registration literature, we refer to the first frame in the sequence (for which a contour is initially provided) as the static image. Contours are found for subsequent frames by pair-wise matching to the first frame: frame 1 to frame 2, frame 1 to frame 3, etc. A frame for which a contour is being determined is referred to as the moving image.

The method involves finding a transformation from the static-image contour, parameterized by x(s)={x₁(s),x₂(s)}, to a contour x″. in a moving image such that the contours correspond to the same anatomical location in both (x′ corresponds to an intermediate contour which will be described). For our purposes, we assume these closed curves are continuous (e.g., piecewise splines) and that their parameterization is normalized such that sε[0, 1]. The point x(0) (equivalently, x(1)) is not arbitrary and is initially picked by the human operator; this will be necessary for region extraction (Section 1.2.1).

The method defines a contour swath as a wide strip extracted from an image along a contour. Each column of the swath is sampled from the image along a line of fixed length w centered at x(sⱼ) and oriented along the vector x(sⱼ)−O, where O is some origin as shown in FIG. 15A. To generate the complete swath, a series of columns is extracted by letting sⱼ=(j−1)/m for j=1 . . . m for m sample points. Hence, pixels in a swath are indexed by (i,j) with iε[(1−w)/2, (w−1)/2] and j=1 . . . m FIG. 15B. The swath associated with a contour a is denoted by Sₐ.

The method defines * as columnwise cross-correlation between a pair of swaths; i.e., if Sₐ=S_{b}*S_{c}, then column j of Sₐ is the sliding dot product between column j of S_{b} with column j of S_{c}.

The method defines the function Q(,) as a swath-similarity metric, i.e., Q(Sₐ, S_{b}). This may be any one of a number of registration metrics, e.g., normalized cross-correlation or an ultrasound-specific metric such as CD₂ [2] or CD_{2bis} [1]. The method assumes these metrics to be maximal for identical swaths.

Section 1.1.1.1 Rigid Matching

Starting with static-image contour x, the following rigid transformations are modeled to match the contour to the moving image: ±x translation, ±y translation, ± rotation, and ± dilation. We assume these transformations T_{1 . . . 8} have associated ΔT_{1 . . . 8} which decide the granularity of the matching process (e.g., 1 pixel or 1 degree). The method proceeds in a gradient ascent as follows:
1. Initialize x′←x. Initialize a transformation list TL 0.
2. Extract Sₓ from the static image. A swath width of ˜15 pixels is appropriate for the scale of most IVUS images. The length of the swath may be the length in pixels of x.
3. Extract S_{x′}, from the moving image. Its width and length should match that of S. The quality of the current match is q=Q(Sₓ, S_{x′})
4.

$$Find \hat{k} = {arg max}_{k = 1. .8} Q ( {S}_{x} , {S}_{{T}_{k ( + )}} ) ,$$

where Tₖ indicates a transformation of type k of magnitude ΔTₖ. The origin for rotation and dilation is the center of mass of S_{x′}, (defined as

$$\{ \frac{1}{‖ {x}_{1}^{′} ‖} ∮ {x}_{1}^{′} ⅆ s , \frac{1}{‖ {x}_{2}^{′} ‖} ∮ {x}_{2}^{′} ⅆ s \}$$

where ∥·∥ represents arclength).
If Q(S, Sₓ, S_{T}_{_{R}}_{(x′)})≦q, then terminate. Otherwise, append Tto TL, let x′←T_{Z,900} (x′), and go to Step 3.

The output of this process is a list TL of applied transformations as well as the final transformed contour x′. This process is guaranteed to terminate as q strictly increases with each iteration. Comparing the transformations applied to a series of frames is useful for statistical analyses of gross motion occurring in the images, e.g., in order to assess vessel wall dilation or relative catheter/vessel rotation over the cardiac cycle (though some of these measurements are invalid if the sequence has previously been gated).

Section 1.1.1.2 Elastic Matching

Given the contour x′, which is itself a rigid transformation of the initial contour x, we may deform x′ elastically in order for it to better conform to the image features relating to x (which is usually manually-drawn). The output of this elastic matching step is a refined contour, x″.

For any contour a, the method defines a contour energy function,

$$E ( a ) = ∮ \{ \begin{matrix}α {\vert {a}_{2} \vert}^{2} + β {\vert {a}_{ss} \vert}^{2} + {γ}_{1} {\vert {I}^{1} ( a ) \vert}^{2} + {γ}_{2} {\vert {I}_{{σ}_{2}}^{2} ( a ) \vert}^{2} + \\ {γ}_{3} {\vert {I}_{h . , h . {σ}_{3}}^{2} ( a ) \vert}^{2}\end{matrix} \} ⅆ s$$

where aₛ and aₛₛ indicate first and second derivatives. By manipulating x′ we seek to maximize E(x′) in order to produce x″. In general this is accomplished using standard deformable-model techniques [4, 5, 6], but some exceptions will be noted later. The coefficients α and β control tension and curvature, which in our context may be used to control the desired accuracy versus smoothness of the contour. The remaining coefficients γ₁, γ₂, and γ₃ weight the influence of functions I¹, I², and I³, which respectively account for elastic deformation between the static- and moving-image features, provide temporal continuity of the contours (i.e., restrict x″ to bear similarity to x′), and take into account statistical differences between regions on opposite sides of a contour. For consistency with the rigid registration step, these energy terms will be defined to be maximal in regions that should attract the contour.

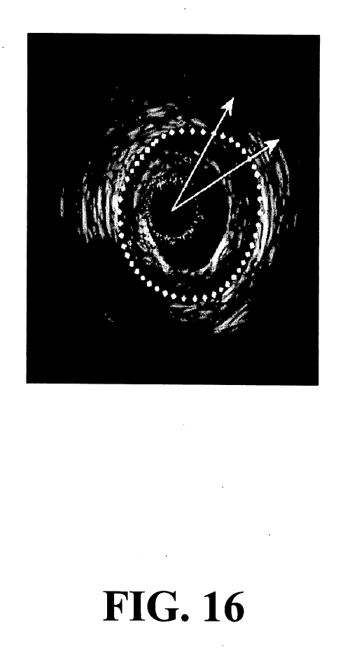

The primary difference between the method described here and standard snakes is that we impose one additional constraint. Namely, the method models deformations as motions strictly toward or away from the catheter in order to enforce that any ray drawn from the catheter outward intersects the contour only once as shown in FIG. 16. To guarantee this, the contour is deformed in the rectangular domain of S_{x′} with its origin O being the center of the catheter and x′ being the output of the rigid matching step. (The “contour” is no longer such, but a periodic curve on the horizontal axis of the swath.) Points along the discretized contour are constrained to move only vertically in this domain.

A point of notation: the term “I_{σ}_{₂}² (a)” refers to function I² evaluated at a with parameter σ₂, where a is itself a vector function of s and returns a 2-D point in the swath domain. To simplify our discussion, S_{x′} will be assumed to have rows on [₋p, +p] and columns on [1,m] (where p is the half-height and m the width of the swath, FIG. 15B). Functions I¹, I², and I³ will be defined as fields on the same domain. It remains to define these functions.

Contour Feature Matching, I¹

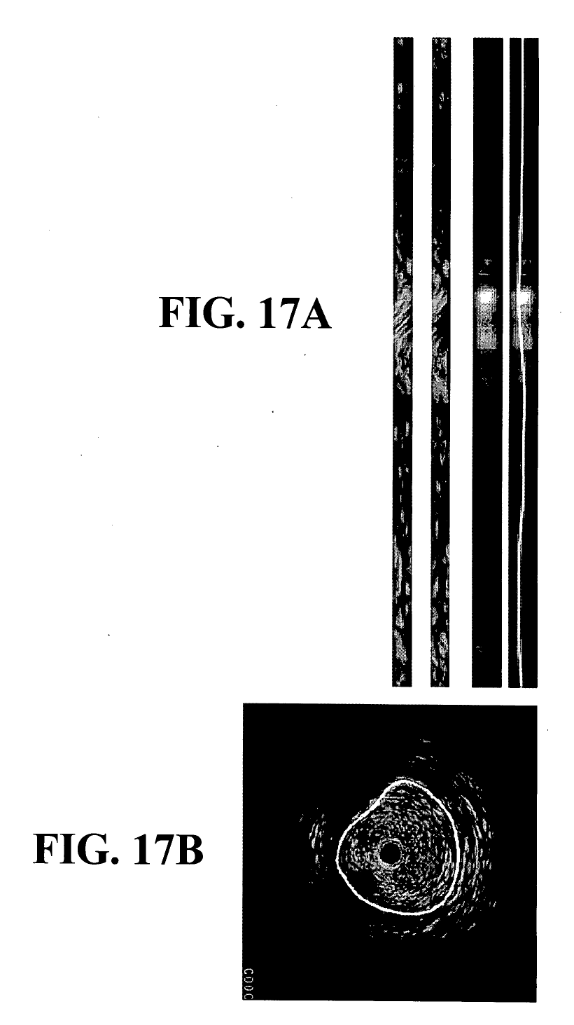

This constraint is the primary means by which the contour seeks similar image features from the static to the moving image. While the search space in non-rigid registration tasks is often very large, we are able to limit it to a smaller, more efficient space under the constraints of contour tracking using the operator. Let
I¹=Sₓ*S_{x′} (1.2)
where Sₓ is the swath around the static-image contour. Note that in image space, this corresponds to a radial correlation operation. Under ideal conditions, the result of this operation is a correlation image which is maximal along its middle row; it is easy to see why this is the case if S_{x′}=Sₓ. Deviations from this condition present non-centered ridges which are sought by the deforming contour function FIG. 17. These ridges will appear higher or lower in the image depending on whether the elastic deformation is occurring toward or away from the catheter. Strong image features will generate strong ridges; shadowed regions will produce little response and hence be interpolated through by the snake without considering these as a special case. In this way, elastically-deforming contour boundaries are efficiently sought in the presence of noise and imaging artifacts.

Shape Prior, Iᵣ²(a)

This constraint forces the contour x″ to bear similarity to the prior contour x′ (which as we recall differs from the static-image contour only by rigid transformations). This is accomplished by centering an energy ridge around the position of the x′ contour. This may be a Gaussian function, in which case this is equivalent to

$${I}_{{σ}_{2}}^{2} ( i , j ) = \frac{1}{{σ}_{2} \sqrt{2 π}} exp ( - \frac{{i}^{2}}{2 {σ}_{2}^{2}} ) \qquad ( 1.3 )$$

The parameter σ₂ may be used to adjust the steepness of this function; if the images are low-motion, increasing σ₂ will prevent the contour from deforming excessively.

Region Feature Matching, Iₕ_{_{•,}}_{,h}_{_{∘,}}_{,σ}₃³

If knowledge of the regions on the inside and outside of the contour is known beforehand, it is possible to use regional statistics to influence the deforming contour. While a number of choices are available in this area, we have found histogram statistics to be effective. Now let h_{•} and h_{∘} be the normalized histograms of the region on the interior and exterior of the static-image contour respectively. A probability may then be developed that a particular grey-level belongs to h_{•} and h_{∘} Intuitively, it is expected that as the contour moves into one of these regions (inappropriately), it will encounter a greater number of grey levels associated with only one of these distributions.

If h_{•} and h_{∘} were true distributions (as opposed to discrete representations), this

$$\begin{matrix}{I}_{{h}_{•} , {h}_{•} , {σ}_{3}}^{3} ( i , j ) = 1 - 4 {( \frac{1}{2} - {P}_{•} [ {Sx}^{′} ( i , j ) ] )}^{2} & ( 1.4 ) \\ where & \\ {P}_{•} ( g ) = \frac{{h}_{•} ( g )}{{h}_{•} ( g ) + {h}_{•} ( g )} & ( 1.5 )\end{matrix}$$

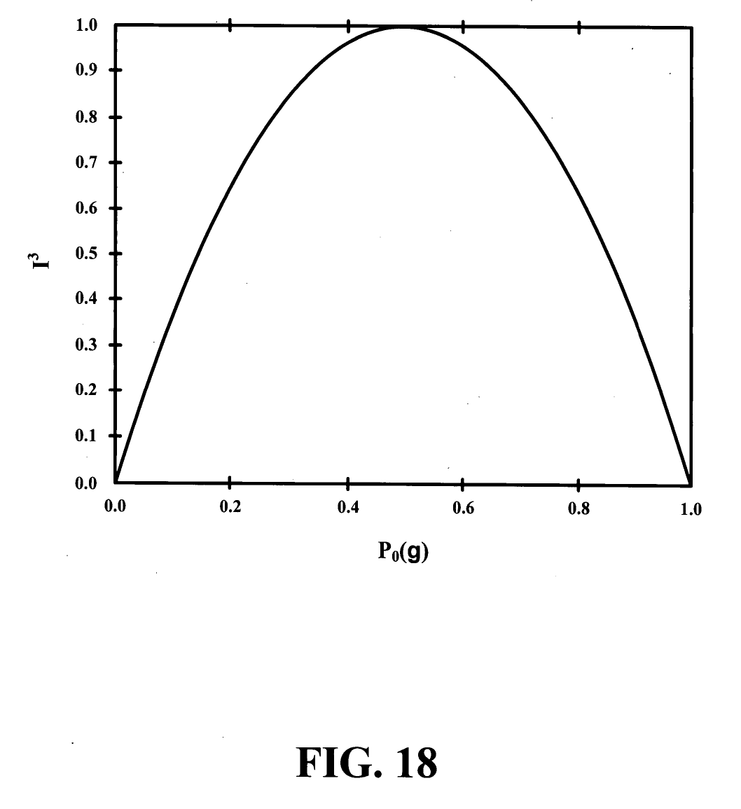

Equation 1.4 is maximal (=1) if the grey level at a point i,j is equiprobabilistic with respect to h_{•} and h_{∘} a shown in FIG. 18. However, as IVUS histograms are often highly discontinuous, P_{•} is unreliable as stated. We define

$${\hat{h}}_{•} ( g ) = {\sum }_{{g}_{0} ∈ G} {h}_{•} ( {g}_{0} ) ( {g}_{0} , g , {σ}_{3} ) \qquad ( 1.6 )$$

where G(x,μ,σ) is the standard normalized Gaussian function evaluated at x, G is the set of all grey levels, and σ₃ is a smoothing parameter (e.g., σ₃=2). This is essentially a kernel density estimator. Now define ĥ_{∘}similarly and substitute ĥ_{●} and ĥ_{∘} into Equation 1.5 in order to make Equation 1.4 more reliable when applied to real data.

As Equation 1.4 is minimal for grey levels which are likely to occur in only one of the distributions and maximal for grey levels which are common to both, our active contour will avoid encroaching inappropriately into areas dominated by a single distribution. In the worst case, h_{•} and h_{∘} will be identical; however, in this case I³ will have no effect on the maximization process as it will be a constant function.

Normalization

As I² and I³ are normalized as presented here (i.e., their ranges do not vary for images with different grey-level properties), we may also apply normalization to I¹ such that γ₁ need not be adjusted for sequences acquired from different sources. In practice, we achieve this by adjusting the values in I′ to zero mean and unit variance.

Reparameterization

As stated, the x(0) point along the original ground-truth contour is picked by the human operator. However, as the goal of the method so far has been to segment the equivalent contour in the moving image, it is not necessarily the case that the point x″(0) corresponds to x(0) anatomically when the rigid and elastic segmentation steps are complete (although they are usually very close). To achieve this, the swaths Sₓ and S_{x″} are compared with the registration metric Q (Section 1.1.1.1) and the starting point of the x″ parameterization is relocated iteratively until Q is maximized. In swath space, this corresponds to sliding S_{x′} left or right (i.e., with wrapping edges) with respect to Sₓ until the maximum is reached. This may be performed in a gradient-ascent manner that in the majority of cases converges in less than 10 iterations with 1-pixel granularity.

Section 1.2 Enhancement Detection

Two steps for tracking a boundary-of-interest throughout a CE-IVUS sequence have described: frame gating followed by hybrid rigid/elastic contour matching. It remains to describe how to employ this in order to track a particular region-of-interest over time and detect the changes occurring in this region.

Section 1.2.1 Region Extraction

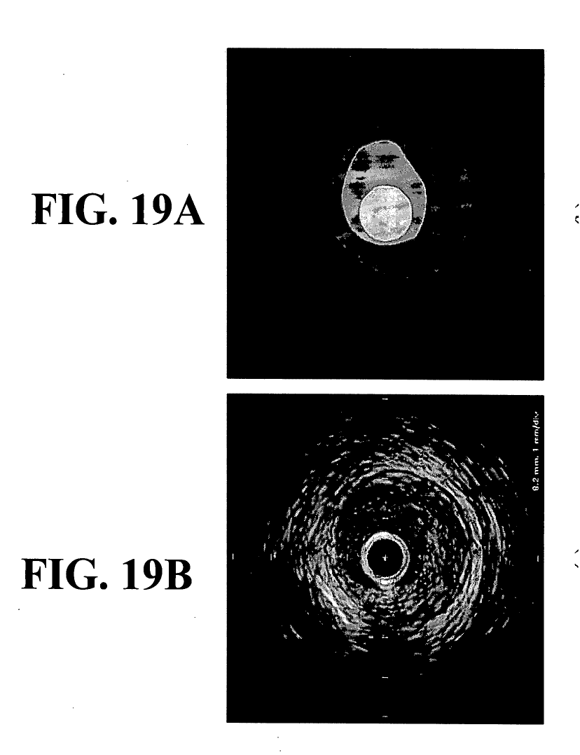

Given the series of gated frames F_{1 . . . n}, contour tracking is used to provide a series of contours on the inside c_{1 . . . n}ⁱⁿ and outside c_{1 . . . n}^{out} of the region of interest. For our purposes, the ROI typically consists of the intimo-medial region, i.e., the inner border is the luminal edge and the outer border is the media/adventitia interface. However, if the adventitia is clearly-defined, this may also be segmented. As described, in the initial contours (c₁ⁱⁿ and c₁^{out}) are provided by the human operator. However, in practice, instead of providing only these initial contours, a 5-region mask is requested FIG. 19 which labels each pixel in the initial frame according to its membership: whether it belongs to the catheter, the lumen, the intima/media, the adventitia, or the outer non-data region of the frame. In this way if, for example, the luminal border is being tracked, the pixels in the lumen and intima/media regions may be used to calculate regional statistics to aid in segmentation (such as h_{•} and h_{∘}, Section 1.1.1.2). In addition, the points c₁ⁱⁿ(0) and c₁^{out}(0) are chosen (manually) such that they correspond to the same point on the interior and exterior of the region of interest. This could also be automated: it is usually sufficient that c₁^{out}(0) is approximately collinear with c₁ⁱⁿ(0) on a ray drawn from the catheter center. Given that these inner and outer boundary points are chosen in the contours on the first frame, and the reparameterization step (Section 1.1.1.2) ensures that the anatomical correspondence of these points is maintained in the moving images, our contours not only provide segmentations, but are also parameterized such that any point c₁^{in/out}(s₀) (i.e., the ground-truth points on the static-image contours) corresponds to the same anatomical point on cᵢ^{in/out} (s₀) for s₀ε[0, 1] and i>1. This fact is critical for region extraction, as follows.

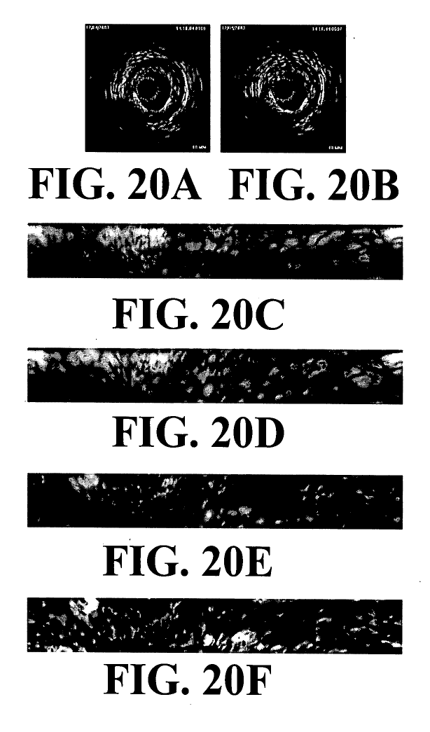

Given a pair of contours for a single frame, we now extract the region between these contours into a rectangular, swath-like domain where analyses become more practical as shown in FIG. 20. The width and height in pixels of these regions may be fixed to ensure comparability; for example, their height may be the maximal distance between c₁ⁱⁿ and c₁^{out} and their width may be the length, in pixels, of c₁^{out}. For each IVUS frame F_{1 . . . n} we then have an associated region image R_{1 . . . n}. A specific point in one region, Rₖ(i,j), corresponds to the same anatomical location as the same point Rₗ(i,j) in another region, given that the underlying contours are anatomically correspondent. As these regions may be mapped into and out of their respective IVUS frames, this achieves the pixel-level correspondence between IVUS regions-of interest that was the ultimate goal of the contour-tracking step. However, we note that one factor that could violate this correspondence is non-uniform dilation or contraction of the vessel wall; as we sample the contour splines uniformly from the static image to the moving image, alignment of the regions in this situation could degrade. While this could be accounted for with a nonlinear reparameterization of the moving-image splines, we have not witnessed cases where this effect is significant.

Section 1.2.2 Difference Imaging

Given the set of region images encompassing our sequence, R_{1 . . . n} it is necessary to assess the changes in this ROI over time. We let τ be the frame immediately prior to the appearance of contrast agent in the lumen. Frames 1 to τ are considered pre-injection, from τ+1 to n are considered post-injection. A pre-injection baseline is calculated by taking the mean region image over this time period,

$${pre}_{} ( i , j ) = \frac{1}{τ} {\sum }_{k = 1}^{τ} {k}_{} ( i , j ) \qquad ( 1.7 )$$

For later purposes, a standard deviation image is also found,

$${pre}_{} ( i , j ) = \sqrt{\frac{1}{τ - 1} {\sum }_{k = 1}^{τ} {[ {k}_{} ( i , j ) - {pre}_{} ( i , j ) ]}^{2}} \qquad ( 1.8 )$$

For the complete sequence of regions, two types of difference images may be derived. The first is a raw difference: [symbol]
^{raw}(i,j)= [symbol] (i,j)− [symbol] ₐᵥₑ(i,j) (1.9)
The second is a difference measured in standard deviations:

$${k}_{std}^{} ( i , j ) = \frac{{k}_{} ( i , j ) - {pre}_{} ( i , j )}{{pre}_{} ( i , j )} \qquad ( 1.10 )$$

In both cases, k=1 . . . n and negative values are thresholded to 0.

In principal, if enhancement due to contrast perfusion occurs, subtracting an unenhanced (pre-injection) image from an enhanced (post-injection) image will result in positive values in those areas of the difference image where enhancement is present FIG. 20E. This concept is expressed by D^{raw}. However, we expect some noise to occur in the pre-injection sequence; the image Sₚᵣₑ models the variability in each pixel due to this noise, and D^{std} measures differences above the noise level FIG. 20F. Comparing these images, we see that the variance-modeled image exhibits greater contrast between enhanced and non-enhanced areas than the raw difference image.

Section 1.2.3 Quantification & Visualization

A set of visualizations are created and statistical analyses are performed on the enhancement data resulting from the previous operations. Due to the preliminary nature of this work, the interpretation of these results is, for now, left to the examiner.

In the case of visualizing raw enhancement data, a threshold T_{raw} is set by the examiner in order to ignore low-order enhancement due to noise (e.g., <30 grey levels). Similarly, for the standard-deviation data, a threshold T_{std} may be set in order to ignore pixels in a region whose values are lower than a certain bound (e.g., <2 standard deviations). Though in either case, these thresholds may be set to 0. Visualizations are created by mapping the difference-image regions D^{raw} and D^{std} into the domain of the original IVUS images and overlaying them in a standard manner (e.g., using color-mapping) so that enhancement may be viewed in its anatomical context.

Enhancement is quantified over time by the following five statistics, which are calculated only after the rectangular region images (i.e., D^{raw} and D^{std}) have been transformed back to the domain of the original IVUS frames. We let mₖ= [symbol] ^{raw/std}|, where |·| denotes area in pixels. For clarity, we will assume that the set of all pixels in a region in image k are indexed from 1 to mₖ.

1. Mean Unthresholded Enhancement in ROI (MUEIR)

This is a gross measure of the change in mean intensity in the ROI over time. While this tends not to indicate false positives in practice (i.e., it will not increase when no enhancement is present), the fact that the enhancement effect tends to be small compared to the entire ROI implies that it may be difficult to detect by this measure. We define MUEIR as:

$${MUEIR}_{k} = \frac{1}{{m}_{k}} {\sum }_{i = 1}^{{m}_{k}} {k}_{raw}^{} ( i ) \qquad ( 1.11 )$$

2. Area of Enhancement above Grey-level Threshold (AGLT)

The value AGLTₖ indicates the area in pixels² of all pixels in [symbol] ^{raw} with a value above T_{raw}.

3. Area of Enhancement above Grev-level Threshold, Fraction of ROI (AGLTF)

This is simply AGLTFₖ=AGLTₖ/mₖ; this may be more reliable than the previous statistic since we expect the area of the ROI to change slightly from one frame to the next.

4. Area of Enhancement above Standard-deviation Threshold (ASDT)

The value ASDTₖ indicates the area in pixels² of all pixels in [symbol] ^{std} with a value above T_{std}.

5. Area of Enhancement above Standard-deviation Threshold, Fraction of ROI (ASDTF)

This is simply ASDTFₖ=AASDTₖ/mₖ. The same reasoning applies to this statistic as to AGLTF.

For reference, these statistics are also summarized in Table 1.1.

TABLE 1.1
Enhancement Metrics and Associated Acronyms

| | |
|---|---|
| MUEIR | Mean unthresholded enhancement in ROI |
| AGLT | Area of enhancement above grey-level threshold |
| AGLTF | AGLT as a fraction of the ROI area |
| ASDT | Area of enhancement above standard-deviation threshold |
| ASDTF | ASDT as a fraction of the ROI area |

An additional statistic, average enhancement per enhanced pixel or AEPEP, which we employed in an earlier method and reported in some publications, has been superseded and will not be discussed here.

While these are per-frame statistics, summary statistics (e.g., mean and standard deviation) for each of these measures may be calculated for the pre-injection and post-injection frame sets separately. If there is a significant difference in mean MUEIR, for instance, this could indicate that enhancement has occurred.

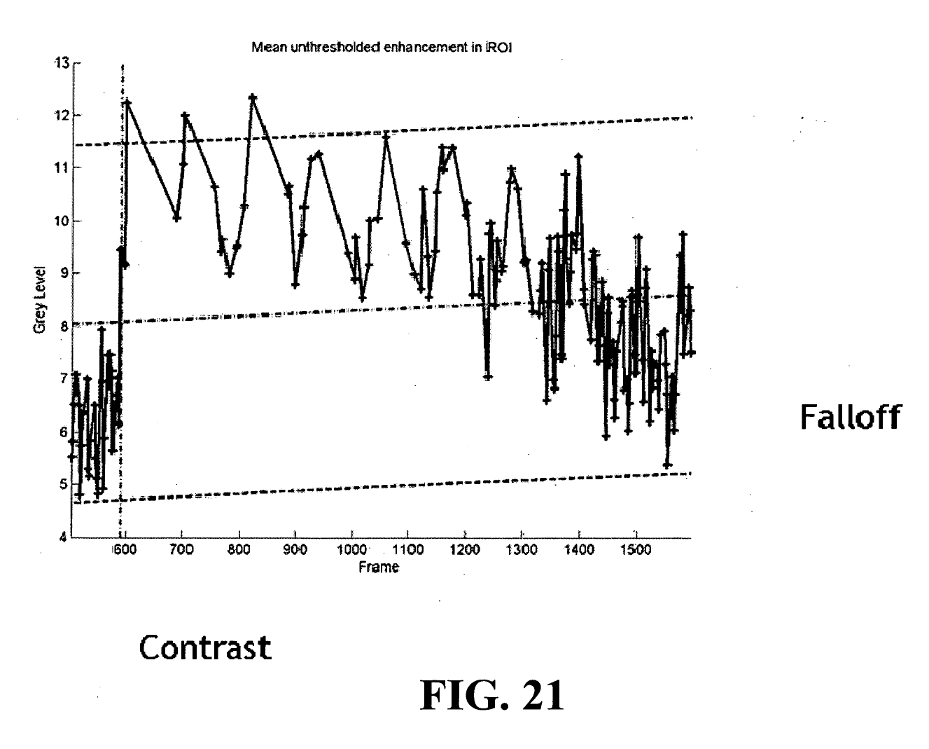

The present invention also relates to a method for differentiating ROIs from nonROIs based on a ratio of a falloff rate in a vessel lumen to a falloff rate in a non-luminal area. The ratio can also be used to differentiate non-luminal plaque and adventitia. Such falloff ratios provide an external reference and has a significant value in identifying active plaques. This data represents another measure of vulnerability which includes the ratio of the falloff of the mean enhancement in the lumen over non-luminal area and similarly for non-luminal to plaque and adventitia. This measure will differentiate two plaques with the same fall off rate in the lumen but different falloff rate in the plaque or adventitia. Imaging two plaques with the same falloff rate in the lumen, but different falloff rates in the plaque and/or adventitia are clear differentiating features of the plaque and/or adventitia. Alternatively, similar falloff rates in two plaques having different luminal falloff rates are clear differentiating features. The inventors believe that lower (luminal versus plaque) falloff ratios represent better predictors of vulnerable plaques. The inventors also believe that lower adventitial versus plaque falloff rates represent better predictors of vulnerable plaques. Referring now to FIG. 21, a plot of a falloff rate of a measures signal of a contrast agent is shown. The data shows that after contrast agent injection, the contrast enhancement falloff at a measurable rate. By measuring the falloff rate of contrast enhancement in a luminal region versus a non-luminal region or in a plaque region versus an adventitial region, plaque can be classified. This data represents another measure of vulnerability which includes the ratio of the falloff of the mean enhancement in the lumen over non-luminal area and similarly for non-luminal to plaque and adventitia.

### REFERENCES CITED IN DIFFERENCE IMAGING FOR MICROBUBBLE CONTRAST DETECTION

[1] D. Boukerroui, J. A. Noble, and M. Brady. Velocity estimation in ultrasound images: A block matching approach. In Inf Process Med Imaging, pages 586-598, 2003.
[2] B. Cohen and I. Dinstein. New maximum likelihood motion estimation schemes for noisy ultrasound images. Pattern Recogn, 35(2):455-463, February 2002.
[3] M. Isard and A. Blake. CONDENSATION: Conditional density propagation for visual tracking. Int J Comput Vision, 29(1):5 28, 1998.
[4] J. Ivins and J. Porrill. Everything you always wanted to know about snakes (but were afraid to ask). AIVRU technical memo, University of Sheffield, July 1993.
[5] M. Kass, A. Witkin, and D. Terzopoulos. Snakes: Active contour models. Int J Comput Vision, 1(4):321331, 1987.
[6] D. Metaxas and I. A. Kakadiaris. Elastically adaptive deformable models. IEEE T Pattern Analysis & Machine Intelligence, 24(10):1310-1321, October 2002.

### F. Methods for Quantification and Visualization of Micro-Vascularization Density of Vasa Vasorum

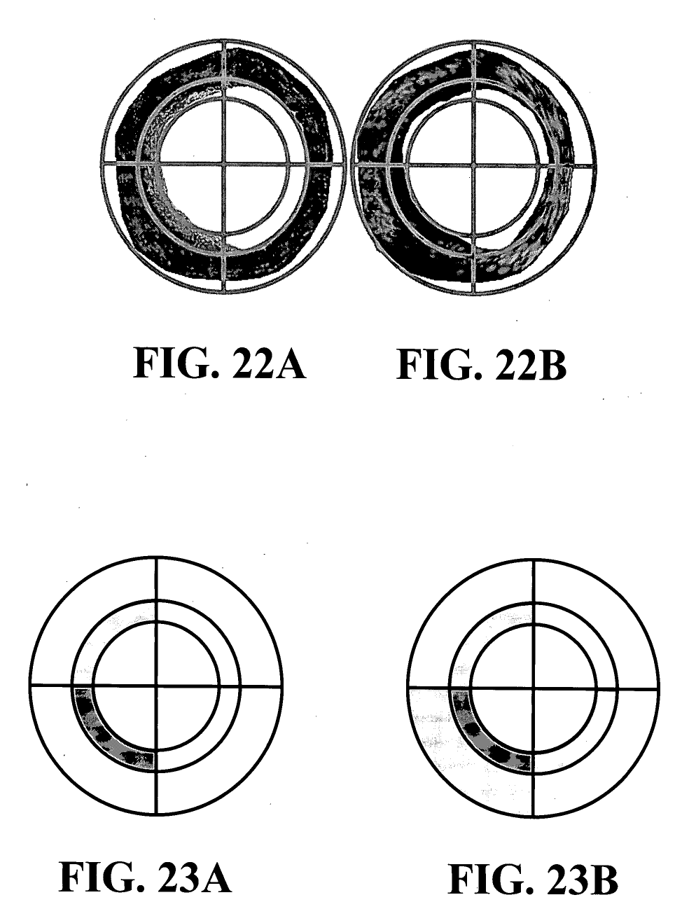

The present invention also relates of a method of visualizing micro-vascularized plaque (a plaque including vasa vasorum) and adventitia segments of a vessel in an animal or human body. The method includes the step of dividing an IVUS image of a vessel and an histology image of a vessel into 4 quadrants and segmenting the images into 8 parts as shown in FIG. 22A or 22B, respectively. Next, a vasa vasorum (VV) density is assessed for each image to allow characterization of plaque as shown in FIGS. 23A and 23B to determine its vulnerability.

Another method includes the step of visualizing the VV density associated with a pullback sequence of IVUS images from a ROI of a vessel. The images are used to construct a map of the plaque VV density in the ROI and a map of the adventitia VV in the ROI. During the pullback, the images are segmented and then divided into four (4) quadrants. Once the sequence of plaque and adventitia images have been segmented and divided, a vasa vasorum (VV) density in each quadrant is assessed. Quadrant maps can be then be stacked for volumetric analysis of plaques or artery segments.

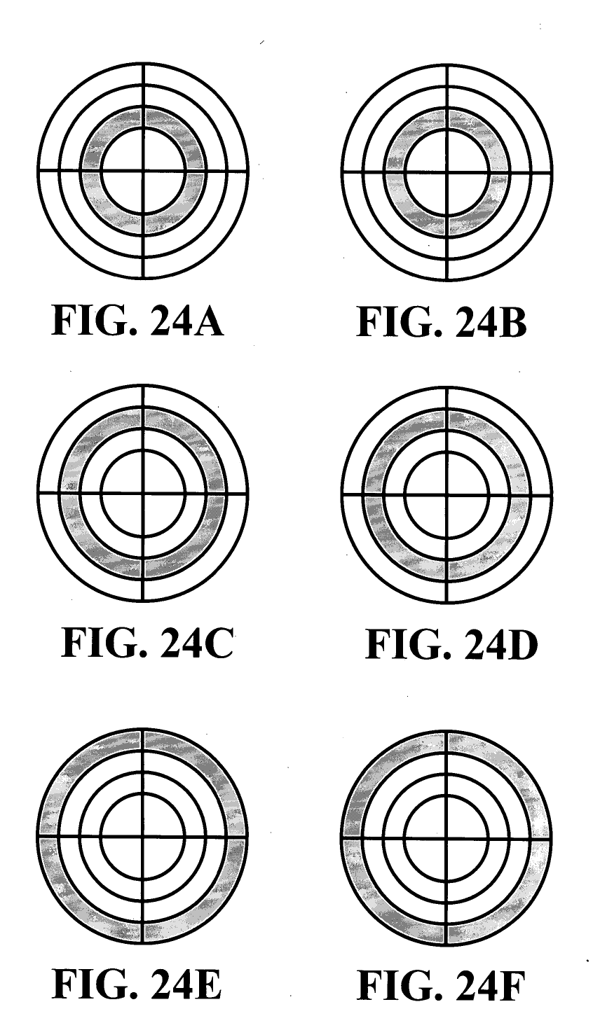

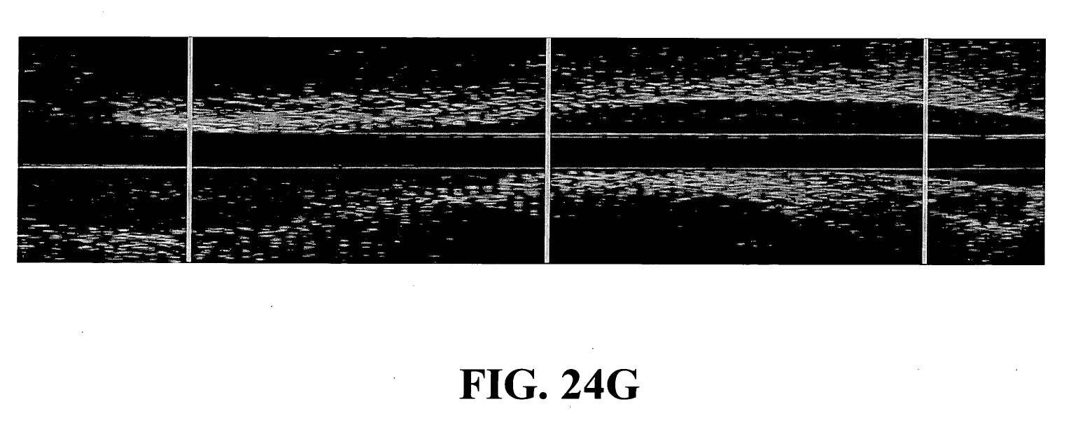

This method for visualizing VV density includes the following steps: (1) after collecting all the images from the pullback, (2) each IVUS frame is divided into quadrants and (3) the VV density at each quadrant is assessed using the methods described herein. The method also includes the step of developing quadrant maps. The quadrant maps can then be stacked from volumetric analysis. Referring now to FIGS. 24A-F, the method graphically illustrates the construction of vasa vasorum (VV) density maps for proximal plaque and adventitia, for medial plaque and adventitia and for distal plaque and adventitia, respectively.

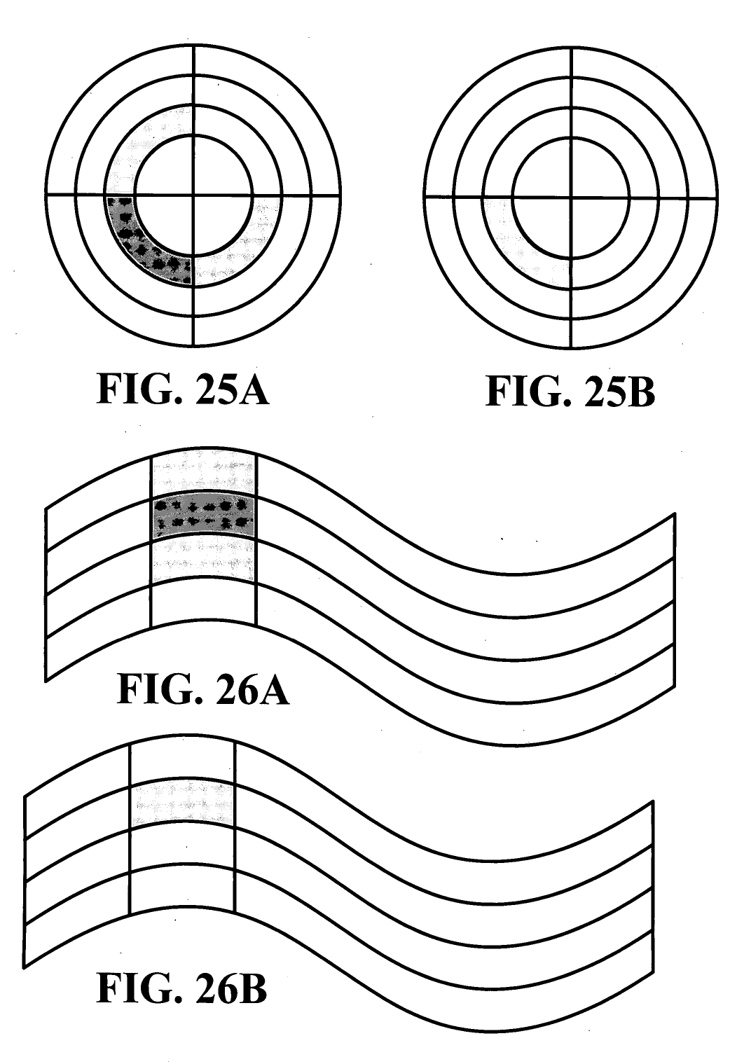

The plaque or adventitia of a complete vessel segment may be summarized by a 12-sector map as shown in FIGS. 25A&B. FIG. 25A shows a plaque vasa vasorum (VV) density map having a high vasa vasorum (VV) density value in a proximal plaque. FIG. 25B shows an adventitial vasa vasorum (VV) density map having a moderate vasa vasorum (VV) density in a proximal adventitia. The 12-sector map can also be presented as an unfolded vessel as shown in FIGS. 26A&B.

Another measure of vulnerability include the ratio of falloff in the lumen over non-luminal area. In addition, the ratio of falloff in the lumen over plaque area and the ratio of falloff in the lumen over adventitia area. For example, two plaques may have the same fall off rate in the lumen but different falloff rate in the plaque or adventitia. Similar falloff rates in two plaques may mean different things if their luminal fall off is not the same.

### G. Methods/Protocols for Contrast Enhanced IVUS Imaging

This portion of the specification describes a method for imaging vulnerable plaque or other regions-of-interest (ROIs) using contrast enhanced IVUS imaging sometimes referred to herein as CEIVUS pronounced SEEVUS. The inventors have found that although a specific external contrast agent can be used, blood itself can act as the contrast agent, whether in static flow or augmented flow.

Five methods or clinical protocols are described for obtaining information on vulnerable plaque. Protocol 1 includes the steps of positioning a catheter at a site to be imaged, holding the catheter stationary at the site, and difference imaging the site, where grey-level difference imaging is used interchangeably with RF-based detection of micro-bubble contrast agents introduced via i.v. injection.

Protocol 2 includes the steps of positioning a catheter at a site to be imaged, holding the catheter stationary at the site, and difference imaging, where grey-level difference imaging is used interchangeably with RF-based detection of micro-bubble contrast agents introduced via i.v. injection and transthorasic excitation. The specifics of transthorasic excitation are as follows: (1) simultaneously with contrast injection, ultrasound acoustic power of 0.6 mechanical index is delivered via a transthorascic transducer (2.5 MHz) towards the left main in order to sonicate the delivered micro-bubbles and (2) immediately after the passage of the contrast agent, which is detected by an intra-coronary ultrasound probe allowing enhanced observations of an entire plaque and adventitia. The procedure also enhances an luminal-intimal boundary allowing clear definition of inner borders of an coronary arterial wall.

Protocol 3 includes the steps of positioning a catheter at a site to be imaged, holding the catheter stationary at the site, and difference imaging, where grey-level difference imaging is used interchangeably with RF-based detection of micro-bubble contrast agents, using intra-coronary injection and reference segment.

Protocol 4 includes the steps of positioning a catheter at a site to be imaged, holding the catheter stationary at the site, and difference imaging, where grey-level difference imaging is used interchangeably with RF-based detection of micro-bubble contrast agents, using intra-coronary injection and adenosine.

Protocol 5, which can be performed with any of the above four protocols (1-4), includes the step of pullback imaging with RF blood detection.

Protocol 1 is a method including the step of positioning an IVUS imaging catheter in an artery to be imaged. After positioning the catheter, pulling back the catheter until a culprit segment or region-of-interest (ROI) segment and a reference segment are identified. After identifying the ROI segment and reference segment, the catheter is re-positioned adjacent the culprit or ROI segment. Images are then collected at a first image or frame collection rate for a first period of time, generally on the order of 30 seconds (30 s). The catheter is then repositioned or moved adjacent the reference segment. Images are collected at an second image or frame rate for a second period of time, generally on the order of 30 seconds (30 s). A contrast agent is then intravenous (iv) injected into the patient and images are collected at a third image or frame collection rate for a third period of time, generally on the order of 60 seconds (60 s). The catheter is then re-positioned or moved to the culprit segment and images are collected at a fourth image or frame collection rate for a fourth period of time, generally on the order of 30 seconds (30 s). Adenosine is then administered and images are collected at a fifth image or frame collection rate for a fifth period of time, generally on the order of 30 seconds (30 s). The catheter is then removed. The collection rates can be the same or different, but in most embodiments are the same for comparison expediency. The time periods can be the same or different and range from 1 second to 5 minutes. In most application, the time periods range between 15 seconds and 75 seconds.

Protocol 2 is a method including the step of positioning an IVUS imaging catheter in an artery to be imaged. After positioning the catheter, pulling back the catheter until a culprit segment or ROI segment and a reference segment are identified. After identifying the ROI segment and reference segment, the catheter is re-positioned adjacent the culprit or ROI segment. Images are then collected at a first image or frame collection rate for a first period of time, generally on the order of 30 seconds (30 s). The catheter is then repositioned or moved adjacent the reference segment. Images are collected at an second image or frame rate for a second period of time, generally on the order of 30 seconds (30 s). A contrast agent is then intravenous (iv) injected into the artery and images are collected at a third image or frame collection rate for a third period of time, generally on the order of 60 seconds (60 s). The catheter is then re-positioned or moved to the culprit segment and images are collected at a fourth image or frame collection rate for a fourth period of time, generally on the order of 30 seconds (30 s), while collecting images, the contrast agent is excited transthoracically during all or some of the image collection period. Adenosine is then administered and images are collected at a fifth image or frame collection rate for a fifth period of time, generally on the order of 30 seconds (30 s). The catheter is then removed. The collection rates can be the same or different, but in most embodiments are the same for comparison expediency. The time periods can be the same or different and range from 1 second to 5 minutes. In most application, the time periods range between 15 seconds and 75 seconds.

Protocol 3 is a method including the step of positioning an IVUS imaging catheter in an artery to be imaged. After positioning the catheter, pulling back the catheter until a culprit segment or ROI segment and a reference segment are identified. After identifying the ROI segment and reference segment, the catheter is re-positioned adjacent the culprit or ROI segment. Images are then collected at a first image or frame collection rate for a first period of time, generally on the order of 30 seconds (30 s). A first amount of a contrast agent is then injected into the artery, intra-coronary injection and images are collected at an second image or frame rate for a second period of time, generally on the order of 30 seconds (30 s). The catheter is then repositioned or moved adjacent the reference segment and images are collected at an third image or frame rate for a third period of time, generally on the order of 30 seconds (30 s). A second amount of a contrast agent is then injected into the artery, intra-coronary injection and images are collected at a third image or frame collection rate for a third period of time, generally on the order of 30 seconds (30 s). The catheter is then removed.

Protocol 4 is a method including the step of positioning an IVUS imaging catheter in an artery to be imaged. After positioning the catheter, pulling back the catheter until a culprit segment or ROI segment and a reference segment are identified. After identifying the ROI segment and reference segment, the catheter is re-positioned adjacent the culprit or ROI segment. Images are then collected at a first image or frame collection rate for a first period of time, generally on the order of 30 seconds (30 s). A first amount of a contrast agent is then injected into the artery, intra-coronary injection and images are collected at an second image or frame rate for a second period of time, generally on the order of 30 seconds (30 s). Adenosine is then administered and images are collected at a fifth image or frame collection rate for a fifth period of time, generally on the order of 30 seconds (30 s). A second amount of a contrast agent is then injected into the artery, intra-coronary injection and images are collected at a third image or frame collection rate for a third period of time, generally on the order of 30 seconds (30 s). The catheter is then removed.

Protocol 5 is a method including the step of performing standard IVUS pullback study of vessel of interest or performing an IVUS study according to the protocols 1-4 of this invention. Simultaneous with the IVUS study, RF data is collected. An RF-based blood detection routine is then used to localize blood beyond an luminal border (i.e., in the plaque and adventitia) in the un-gated sequence. The un-gated sequence is then analyzed using a gating method to produce a gated sequence. Next, a volumetric reconstruction of vessel for visualization and statistical quantification of structures such as vasa vasorum.

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

We claim:

1. A catheter apparatus comprising:
a nozzle system having exit holes around its periphery adapted to direct jets of an agent through the holes near, immediately proximate or immediately adjacent a portion of a vessel wall of a vessel to be imaged,
a conduit connecting the nozzle system to an external or internal agent reservoir,
at least one electronic flow controller and/or injector adapted to control a flow of the agent from the reservoir through the conduit to the nozzle system out of the holes, and
a digital or analog processing unit adapted to control the controllers.

2. The apparatus of claim 1, further comprising:
an intravascular ultrasound (IVUS) probe connected to an IVUS imaging unit, and
an IVUS digital or analog processing unit adapted to receive and analyze IVUS data before, during and/or after agent injection.

3. The apparatus of claim 1, further comprising:
at least one Doppler element adapted to collect Doppler data connected to a Doppler imaging unit, and
a Doppler digital or analog processing unit adapted to receive and analyze Doppler data before, during and/or after agent injection.

4. A method comprising the steps of:
positioning a probe adjacent a portion of a vessel, where the probe includes an intravascular ultrasound (IVUS) component and a Doppler component,
transmitting a plurality of imaging pulses from the IVUS component into an region-of-interest (ROI),
receiving echoes from the ROI by the IVUS component,
matching or correlating the echoes to the image pulses to estimate a radial position of the stationary probe in the image,
while maintaining the probe in a stationary orientation, switching to pulsed Doppler component;
detecting Doppler signals evidencing flow within the ROI where a suspected plaque, microvascularization or vasa vasorum site is located.

5. A radio-frequency (RF) detection, analysis and quantification method for IVUS comprising the steps of
obtaining RF-based IVUS data with or without an external contrast agent, and
analyzing the RF-based IVUS data to determine features of the vessel.

6. The method of claim 5, wherein the obtaining step comprises:
positioning an IVUS catheter at a maximally-stenotic point of a suspect plaque; and
recording RF data, while the IVUS catheter held stationary in the vessel.

7. The method of claim 6, wherein blood acts as the contrast agent and data is collected for a number of cardiac cycles.

8. The method of claim 6, wherein the obtaining step further comprises:
introducing a contrast agent, and
recording RF data before, during, and after contrast agent introduction.

9. The method of claim 5, wherein the obtaining step comprises:
positioning an IVUS catheter at a maximally-stenotic point of a suspect plaque; and
recording RF data as the catheter is being pulled back.

10. The method of claim 9, wherein the obtaining step further comprises:
introducing a contrast agent, and
recording RF data before, during, and after contrast agent introduction as the catheter is being pulled back.

11. A method implemented on a computer for detecting and analysis RF IVUS blood/saline/contrast agent data comprising the steps of:
training an algorithm to produce a optimal feature classifier including the steps of:
obtaining RF IVUS data including a plurality of frames from an appropriate region of interest (ROI) of a vessel, where the ROI evidences the presence of blood, saline and/or contrast agent,
computing a number of features of the ROI,
computing which features result in a best classification for a given classifier,
selecting the given classifier, and
optimizing parameters of the given classifier to produce the optimal feature classifier, and
deploying the algorithm including the steps of:
using the optimal feature classifier to classify the RF IVUS data.

12. A method for frame gating comprising the steps of:
obtaining a sequence of frames representing a time period from a first time t₁ to a second time t₂;
selecting a frame similarity or dissimilarity metric,
generating a frame dissimilarity matrix,
generating a frame-similarity space by applying the metric to the dissimilarity matrix,
generating clusters in the space using a clustering function, and
selecting a particular frame ensemble from the resulting clusters.

13. A method for pullback frame gating comprising the steps of:
obtaining a pullback sequence of frames representing a time period from a first time t₁ to a second time t₂;
selecting a frame similarity or dissimilarity metric,
generating a dissimilarity matrix,
obtaining an initial estimate of a heartrate over the entire recording,
tracing a path along an off-diagonal valley which represents a cardiac cycle length locally at each frame,
applying zn x-shaped filter to find frame pairs associated with both high similarity and low motion,
finding a single-phase associated pair that is at a maximally-stable point in the cardiac cycle, and
using this point for tracing the filtered dissimilarity matrix upward and downward to collect the frames for the gated sequence.

14. A difference imaging-based detection method comprising the steps of:
positioning a catheter at a maximally-stenotic point of a suspect plaque or a region of interest (ROI);
imaging the ROI for a first period of time, generally on the order of 30 seconds, while holding the catheter steadily in place;
injecting a bolus dose of a contrast agent or contrast effect intra-coronarily, proximate the imaging catheter;
imaging the ROI for a second period of time, generally on the order of 30 seconds, again while holding the catheter steadily in place;
image-based frame gating the data to decimate a number of frames in the sequence of image frame collected in steps 2 and 4, providing a stabilized gated sequence with fewer frames than the original;
outlining an inner and outer contours of the ROI in the first frame of the gated sequence, performed either by an operator or an outlining routine;
propagating these contours to the remaining frames in the sequence, in order to provide a segmentation of each frame;
extracting a region between the contours into a rectangular raster in each frame, providing a stabilized space in which inter-frame comparisons of the ROI are to be performed;
averaging a pre-injection ROI images to obtain a pre-injection baseline of the non-contrast ROI;
subtracting the averaged baseline ROI pixel-wise from the pre- and post-injection ROI images to detect differences between the baseline appearance and the pre- and post-contrast appearance, where the pre-injection frames will rarely exhibit any changes as the pre-injection baseline is derived from them; and
mapping the difference-imaged ROIs back into the original IVUS space for visualization and quantification of the changes which occurred due to contrast perfusion.

15. A method comprising the steps of:
performing an intravascular ultrasound (IVUS) pullback analysis of a vessel,
performing optionally pullback frame gating, analyzing each frame in the original or gated sequence for detection and qualification of VV
dividing each image into four (4) quadrants constructing a map of the VV densities in each quadrant, and
visualizing the VV densities associated with the pullback sequence of IVUS images.

16. A clinical method comprising the steps of:
positioning an IVUS imaging catheter in a vessel,
after positioning, pulling back the catheter until a region-of-interest (ROI) segment of the vessel and a reference segment of the vessel are identified.
after identifying the ROI segment and reference segment, re-positioning the catheter adjacent the ROI segment,
collecting ROI images at a first frame collection rate for a first period of time,
repositioning the catheter adjacent the reference segment,
collecting reference images at a second frame collection rate for a second period of time, introducing a contrast agent,
a collecting contrast reference images at a third frame collection rate for a third period of time,
re-positioning the catheter adjacent the ROI segment,
collecting contrast ROI images at a fourth frame collection rate for a fourth period of time,
optionally while collecting images, exciting the contrast agent transthoracically during all or some of the third and/or fourth image collection period,
administering adenosine,
collecting adenosine ROI images at a fifth frame collection rate for a fifth period of time,
removing the catheter is then removed, and
analyzing the images to assess the presence of plaques and/or vasa vasorum (VV) within the ROI segment.
